(19) United States
(12) Patent Application Publication
(10) Pub. No.: US 2014/0278165 A1
(43) Pub. Date: Sep. 18, 2014

(54) SYSTEMS AND METHODS FOR ANALYZING ENERGY CONSUMPTION MODEL DATA

(71) Applicant: Johnson Controls Technology Company, Holland, MI (US)

(72) Inventors: Michael J. Wenzel, Oak Creek, WI (US); Kirk H. Drees, Cedarburg, WI (US); Clay G. Nesler, Brookfield, WI (US)

(73) Assignee: Johnson Controls Technology Company, Holland, MI (US)

(21) Appl. No.: 14/209,234

(22) Filed: Mar. 13, 2014

Related U.S. Application Data
(60) Provisional application No. 61/785,739, filed on Mar. 14, 2013.

(51) Int. Cl. G01R 21/00; G01N 33/00

(52) U.S. Cl. CPC G01R 21/00; G01N 33/00
USPC 702/61; 702/183

(57) ABSTRACT

A building's energy consumption may be modeled using weather data, utility billing data, or other data regarding the building. The resulting model data may be analyzed to detect a shift in the model data, which may indicate the presence of a fault condition. Changes to the model's coefficients that would result from an upgrade, energy conservation measure, or other action may also be used to predict the resulting Energy Star score for the building.

### CROSS-REFERENCE TO RELATED PATENT APPLICATIONS

This application claims the benefit of and priority to U.S. Provisional Patent Application No. 61/785,739, filed Mar. 14, 2013, the entirety of which is incorporated by reference herein.

### BACKGROUND

The present disclosure generally relates to systems and methods for analyzing energy consumption model data.

Many commercial buildings today are equipped with a variety of energy-consuming devices. For example, a commercial building may be equipped with various heating, ventilation, and air conditioning (HVAC) devices that consume energy to regulate the temperature in the building. Other exemplary types of building equipment that consume energy may include lighting fixtures, security equipment, data networking infrastructure, and other such equipment.

The energy efficiency of commercial buildings has become an area of interest in recent years. In many areas of the world, commercial buildings consume a good portion of the generated electricity available on an electric grid. For an energy provider, the energy efficiency of commercial buildings that it services helps to alleviate strains placed on the provider's electrical generation and transmission assets. For a building's operator, energy efficiency corresponds to greater financial savings, since less energy is consumed by the building.

One measure of energy efficiency is an Energy Star score. Originally adopted by the United States Environmental Protection Agency (U.S. EPA), Energy Star scores have since been adopted throughout the world as a standard measure of a building's energy consumption. A building's Energy Star score is typically measured on a scale ranging from 1-100, which indicates the building's energy efficiency relative to similar buildings in its class. For example, a data center with an Energy Star score of 75 is in the seventy fifth percentile among other data centers in its class.

A building's operator may take certain steps to improve the energy efficiency of the building. For example, the building's operator may implement energy conservation measures (ECMs) or correct equipment faults in the building's existing systems. ECMs may involve upgrading the building's equipment to use more energy-efficient equipment or altering how the building's equipment is controlled (e.g., by turning the building's lights off at a certain time, adjusting the building's internal setpoint temperature, etc.). Correcting equipment faults in the building's existing systems also presents another opportunity to reduce the building's energy consumption. For example, a stuck outdoor air valve on a hot day may cause the building to consume more energy than needed to cool the building to a setpoint temperature. However, it remains challenging and difficult to identify potential ways to reduce a building's energy consumption.

### SUMMARY

One embodiment relates to a method for evaluating a fault condition in a building. The method includes generating, by a processing circuit, an energy consumption model for the building. The method also includes using the energy consumption model and input data from different time windows to generate model data. The method further includes analyzing the model data to detect a non-routine change in the model data across the different time windows. The method also includes providing an indication of a potential fault condition based on the non-routine change in the model data being detected.

Another embodiment relates to a system for evaluating a fault condition in a building. The system includes a processing circuit configured to generate an energy consumption model for the building. The processing circuit is also configured to use the energy consumption model and input data from different time windows to generate model data. The processing circuit is further configured to analyze the model data to detect a non-routine change in the model data across the different time windows. The processing circuit is additionally configured to provide an indication of a potential fault condition based on the non-routine change in the model data being detected.

Yet another embodiment relates to a method for determining a change to an energy score of a building. The method includes generating, by a processing circuit, an energy consumption model for the building. The method also includes using the energy consumption model and input data regarding the building to calculate baseline model data, the baseline model data being associated with a baseline energy score. The method further includes receiving an identifier representing a proposed change to the operation of the building, the received identifier being associated with a change to the model data. The method also includes calculating an energy score associated with the proposed change using the baseline model data, the change to the model data associated with the proposed change, and the baseline energy score.

Alternative exemplary embodiments relate to other features and combinations of features as may be generally recited in the claims.

### BRIEF DESCRIPTION OF THE FIGURES

The disclosure will become more fully understood from the following detailed description, taken in conjunction with the accompanying figures, wherein like reference numerals refer to like elements, in which:

FIG. 1 is an illustration of a building data acquisition and analysis system, according to an exemplary embodiment;

FIG. 2 is an illustration of building model parameters, according to one embodiment;

FIG. 3 is a block diagram of a processing circuit configured to model and analyze a building's energy consumption, according to an exemplary embodiment;

FIG. 4 is a flow chart of a process for identifying an equipment fault in a building, according to an exemplary embodiment;

FIG. 5 is a flow chart of a process for using a control chart to identify an equipment fault in a building, according to an exemplary embodiment;

FIG. 6 is a flow chart of a process for using a confidence interval to identify an equipment fault in a building, according to an exemplary embodiment;

FIG. 7 is a flow chart of a process for using hypothesis testing to identify an equipment fault in a building, according to an exemplary embodiment;

FIG. 8 is a flow chart of a process for using recursive residuals to identify an equipment fault in a building, according to an exemplary embodiment; and

FIG. 9 is a flow chart of a process for using a building model to determine an Energy Star score, according to an exemplary embodiment.

### DESCRIPTION

Before turning to the figures, which illustrate the exemplary embodiments in detail, it should be understood that the disclosure is not limited to the details or methodology set forth in the description or illustrated in the figures. It should also be understood that the terminology is for the purpose of description only and should not be regarded as limiting.

According to various aspects of the present disclosure, a building's energy consumption may be modeled in a lean manner using readily available data as inputs to the model. In some embodiments, a building's energy consumption is modeled using the building's utility data (e.g., from a utility that supplies electricity to the building) and weather data for the building's geographic location (e.g., data indicative of historical weather patterns). The model's parameters may also be normalized to allow comparisons to be made between the building and similar buildings. For example, the model's parameters may be normalized using data regarding the building's floor space and compared to other buildings located in the same climate or having the same usage type (e.g., hospitals, university buildings, apartment buildings, etc.). The model's parameters may also be normalized to account for routine weather changes. Thus, the building's energy consumption can be modeled and evaluated without requiring an expensive energy audit or monitoring every aspect of the building via deployed sensors.

In some embodiments, a building's energy consumption model can be used to detect equipment faults. Data from sliding timeframes may be used with the model. The results can be analyzed statistically to detect non-routine changes. In one embodiment, a statistical process control chart may be trained using the variables of the energy consumption model. Statistically significant deviations corresponding to equipment faults can then be detected. In another embodiment, the variables of the energy consumption model may be used to generate a confidence interval. Observations that fall outside of the confidence interval may then be used to identify a potential fault condition. In a further embodiment, hypothesis testing may be used on the coefficients of the energy consumption model to detect non-routine changes in the model. In yet another embodiment, recursive residuals may be generated from the energy consumption model's parameters and analyzed to detect non-routine changes in the model. For example, a statistical process control chart may be generated using the recursive residuals and analyzed to detect non-routine changes.

Techniques are also disclosed to use a building's energy consumption model to analyze the impact of equipment upgrades, improvements, and ECMs on the building's Energy Star score. For example, upgrading the building's heating unit to a more energy efficient model may increase the building's Energy Star score. In various embodiments, a change to the building's energy consumption as a result of an equipment change or implementation of an ECM is determined using the building's energy consumption model. The resulting change may then be mapped to an Energy Star score, allowing the building's operator to quantify the effects of implementing ECMs and equipment changes.

Building Data Acquisition and Analysis

Referring now to FIG. 1, an illustration of a building data acquisition system 100 is shown, according to an exemplary embodiment. Generally, building data acquisition system 100 is configured to record, store, and analyze building data related to a building's energy consumption. In various embodiments, building data for a building may be used to model the building's energy consumption. The resulting model data may then be analyzed to detect fault conditions, analyze the potential impact of change to the operation of the building (e.g., changing how the building's existing equipment is operated, making changes to the equipment itself, etc.), and perform other analytical operations.

As shown, building data acquisition system 100 may include any number of buildings 102-106 (e.g., a first through nth building). Buildings 102-106 may also include any number of different types of buildings, such as various types of commercial buildings. For example, building 102 may be an office building, building 104 may be a manufacturing facility, and building 106 may be a hospitality facility, such as a hotel. Other exemplary buildings in buildings 102-106 may include, but are not limited to, data centers, schools, shipping facilities, and government buildings. Buildings 102-106 may include any combination of the different building types. For example, buildings 102-106 may include ten office buildings, twenty manufacturing facilities, and thirty hospitality facilities.

Buildings 102-106 may be located within the same geographic regions as one another or across different geographic regions. For example, building 102 and building 104 may be located in the same city, while building 106 may be located in a different city. Different levels of granularity may be used to distinguish buildings 102-106 as being located in the same geographic region. For example, geographic regions may be divided by country, state, city, metropolitan area, time zone, zip code, area code, latitude, longitude, growing zone, combinations thereof, or by using any other geographic classification system. According to one embodiment, a building's geographic location may be used as a proxy for its climatic zone. For example, data regarding a building's location in Hawaii may be used to determine that the building is located in a tropical climate.

Buildings 102-106 may be equipped with sensors and other monitoring devices configured to measure building data related to the building's energy consumption. For example, buildings 102-106 may have devices (e.g., computing devices, power meters, etc.) configured to measure the water consumption, energy consumption, and energy demand of the buildings. Other forms of building data may include the measured temperature in the zones of a building, the dimensions of the building (e.g., square footage, etc.), and any other measured value that relates to the building's energy consumption profile. In some cases, building data may also include data used in a building's automation system. For example, building data may also include control parameters, such as temperature set points used to regulate the temperature in a building and timing data used to automatically turn on or off parts of the lighting within the building at various times (e.g., the lights may be turned off in an area of the building at night). In some embodiments, however, a building's energy consumption may be modeled and analyzed without using complex sensor data from the building or control parameters from the building's control system.

According to various embodiments, readily available data may be used to determine and model a building's energy consumption. For example, billing data may be received from a utility 114 (e.g., billing data from the utility) that indicates the building's energy consumption, the financial costs associated with the energy consumption, etc. In keeping with the principles of lean energy analysis described herein, billing data from a utility and/or other forms of readily available data may be used to model and analyze a building's energy consumption. Such an approach may simplify and reduce the cost of performing the energy analysis over approaches that rely heavily on sensor data from a building.

Building data may include data regarding the weather where a building is located. In some embodiments, the weather data may be generated by weather-sensing equipment at buildings 102-106. For example, building 104 may be equipped with temperature sensors that measure the building's external temperature. In some embodiments, building data may include weather data received from a weather data source located in proximity to the building. In further embodiments, building data may include weather data for a typical meteorological year (TMY) received from a historical weather data source 112 (e.g., a computer system of the National Oceanic and Atmospheric Administration or similar data source). In the United States of America, the first set of TMY data was collected between 1948-1980 from various locations throughout the country. A second set of TMY data (TMY2), which also includes data regarding precipitable moisture, was collected between 1961-1990. In addition, a third set of TMY data (TMY3), was collected from many more locations than TMY2 data over the span of 1976-1995. Regardless of the version used, TMY data may be used to compare current conditions to normal or predicted conditions, in some embodiments. In further embodiments, TMY data may be used to predict future conditions of a building (e.g., by using the historical data to predict typical future weather conditions) or future energy consumptions by a building. For example, TMY data may be used to predict an average outdoor temperature change for a building during the upcoming month of March. TMY data may be stored by the building automation systems of buildings 102-106 or data acquisition and analysis service 110 and used to model the heating and cooling needs of buildings 102-106. As used herein, “TMY data” may refer to any version or set of TMY data (e.g., TMY2 data, TMY3 data, etc.).

Network 108 may be any form of computer network that relays information between buildings 102-106 and a data acquisition and analysis service 110. For example, network 108 may include the Internet and/or other types of data networks, such as a local area network (LAN), a wide area network (WAN), a cellular network, satellite network, or other types of data networks. Network 108 may also include any number of computing devices (e.g., computer, servers, routers, network switches, etc.) that are configured to receive and/or transmit data within network 108. Network 108 may further include any number of hardwired and/or wireless connections. For example, building 102 may communicate wirelessly (e.g., via WiFi, ZigBee, cellular, radio, etc.) with a transceiver that is hardwired (e.g., via a fiber optic cable, a CAT5 cable, etc.) to other computing devices in network 108.

Data acquisition and analysis service 110 may be one or more electronic devices connected to network 108 configured to receive building data regarding buildings 102-106 (e.g., either directly from buildings 102-106 or from another computing device connected to network 108). In various embodiments, data acquisition and analysis service 110 may be a computer server (e.g., an FTP server, file sharing server, web server, etc.) or a combination of servers (e.g., a data center, a cloud computing platform, etc.). Data acquisition and analysis service 110 may also include a processing circuit configured to perform the functions described with respect to data acquisition and analysis service 110. The building data may be received by the processing circuit of data acquisition and analysis service 110 periodically, in response to a request for the data from data acquisition and analysis service 110, in response to receiving a request from a client device 116 (e.g., a user operating client device 116 may request that the building data be sent by the computing device), or at any other time.

Data acquisition and analysis service 110 may be configured to model the energy consumption profiles of buildings 102-106 using the received building data. For example, data acquisition and analysis service 110 may utilize lean energy analysis (e.g., using readily available data, such as utility billing data) to model the energy consumptions of buildings 102-106. In some embodiments, data acquisition and analysis service 110 may use the received building data in an inverse building energy model that uses weather data as an independent variable and energy bill data divided by the area of the building as the dependent variable (e.g., energy consumption data that has been normalized based on the building's internal area). In other words, the model may make use of historical weather data to predict the energy costs for the building using lean energy analysis. Data acquisition and analysis service 110 may also generate and provide various reports to client device 116, which may be located within one of buildings 102-106 or at another location.

In other embodiments, data acquisition and analysis service 110 may be implemented at one or more of buildings 102-106. For example, data acquisition and analysis service 110 may be integrated as part of the building automation system of buildings 102-106 (e.g., as part of a distributed implementation). In such a case, building data may be shared by the computing devices in buildings 102-106 that implement the functions of data acquisition and analysis service 110 with one another via network 108. For example, computing devices at buildings 102-106 may be configured to collaboratively share building data regarding their respective building's energy consumption and demand. The sharing of building data among the buildings' respective computing devices may be coordinated by one or more of the devices, or by a remote coordination service. For example, a remote server connected to network 108 may coordinate the sharing of building data among the electronic devices located at buildings 102-106.

Referring now to FIG. 2, an illustration 200 of building model parameters is shown, according to one embodiment. In general, a number of different factors may affect the energy consumption of a building. For example, the outdoor air temperature of the building may affect the building's energy consumption (e.g., to heat or cool the building to a set point temperature). The building's energy consumption profile when cooling the building may also differ from the building's energy consumption profile when heating the building. In some embodiments, the building's energy consumption model may include parameters relating to both heating and cooling the building.

As shown in illustration 200, an x-y plot may be formed with a building's energy consumption (E) plotted along a first axis 202 and the outdoor air temperature (T_{OA}) plotted along a second axis 204. In various embodiments, the building's energy consumption plotted along axis 202 may be an energy consumption (e.g., measured in kWh) or an energy cost associated with the building's energy consumption (e.g., by multiplying the consumption by a cost per consumption value in $/kWh). Such information may be obtained, for example, from billing data for the building from the utility providing the energy to the building. In one embodiment, the outdoor air temperature may be measured for a building using sensors located at or near the building over a particular time period.

A first parameter that may be used to model a building's energy consumption is its base energy load (E_{O}) 206. In general, base energy load 206 corresponds to the energy consumption of the building at any given time that does not change with the outdoor air temperature. For example, base energy load 206 may be a function of the energy consumption of the building's lighting, computer systems, security systems, and other such electronic devices in the building. Since the energy consumption of these devices does not change as a function of the outdoor air temperature, base energy load 206 may be used to represent the portion of the building's energy consumption that is not a function of the outdoor air temperature.

In some embodiments, heating degree day (HDD) and cooling degree day (CDD) values for a building may be calculated by integrating the difference between the outdoor air temperature of the building and a given temperature over a period of time. In one embodiment, the given temperature may be cooling balance point 210 for the building (e.g., to determine a CDD value) or heating balance point 208 for the building (e.g., to determine an HDD value). For example, assume that the cooling balance point for a building is 67° F. In such a case, the CDD value for the building over the course of a month may be calculated as follows:

$$CDD = {\int }^{month} Max \{ 0 , ( {T}_{OA} - 67 ° F . ) \} d t$$

In other embodiments, a set reference temperature may be used to calculate a building's CDD or HDD value instead of the building's actual balance point. For example, a reference temperature of 65° F. may be used as a fixed value to compare with the building's outdoor air temperature. Thus, a CDD or HDD value may generally represent the amount of heating or cooling needed by the building over the time period.

A heating slope (S_{H}) 212 may correspond to the change in energy consumption or energy costs that result when the outdoor air temperature drops below a heating balance point (T_{bH}) 208 (e.g., a breakeven temperature). For example, assume that heating balance point 208 for a building is 55° F. When the outdoor air temperature is at or above 55° F., only energy expenditure equal to base load 206 may be needed to maintain the internal temperature of the building. However, additional energy may be needed if the outdoor air temperature drops below 55° F. (e.g., to provide significant mechanical heating to the interior of the building). As the outdoor air temperature decreases, the amount of energy needed to heat the building likewise increases at a rate corresponding to heating slope 212.

Similar to heating balance point 208, a cooling balance point (T_{bC}) 210 may correspond to the outdoor air temperature at which additional energy beyond base energy load 206 is needed (e.g., the energy needed to provided mechanical cooling to the interior of the building). As the outdoor air temperature rises beyond cooling balance point 210, the amount of energy needed for cooling with also increase at a rate corresponding to cooling slope (S_{C}) 214.

One potential energy consumption model that takes into account the various model parameters illustrated in illustration 200 is as follows:

E=β₀(# days)+β₁(CDD)+β₂(HDD)+ε

where E is the dependent variable representing the energy consumption or cost plotted along axis 202 in illustration 200. β₀ may be a base energy consumption, such as base energy load 206. β₁ may correspond to cooling slope 214 that, when multiplied by the CDD for a particular time, results in an energy consumption or cost attributable to cooling the building. Similarly, β₂ may correspond to heating slope 212 that, when multiplied by the HDD for a particular time, results in an energy consumption or cost attributable to heating the building. The value of E may correspond to the amount of error or noise in the model. In some embodiments, the model may instead model the energy-related costs for the building by multiplying the building's energy consumption by a conversion factor (e.g., by multiplying by a cost factor measured in $/kWh). In further embodiments, the model may be normalized by dividing the model by the internal area of the building. For example, the model may model the normalized energy consumption (e.g., measured in kWh/ft²) or normalized energy cost (e.g., measured in $/ft²).

According to various embodiments, the various parameters used in a building's energy consumption model may be represented as a multidimensional vector. For example, one vector may be defined as a five-dimensional vector as follows:

$${φ}_{m} = [ \begin{matrix}{E}_{0} \\ {S}_{H} \\ {S}_{C} \\ {T}_{bH} \\ {T}_{bC}\end{matrix} ] ∈ {R}^{5}$$

Other energy consumption models having a different number of parameters may also be generated, in other embodiments. For example, assume that the climate where a building is located is such that the building only provides heating or cooling to its internal areas (e.g., a building in Alaska may provide year-round heating to its internal areas, etc.). In such cases, the building may not exhibit either a heating or cooling balance point and a three parameter model may be used to model the building's energy consumption. In another example, assume that a building transitions between supplying heating and cooling at a single balance point (e.g., the building's heating balance point and cooling balance point are equal). In such a case, a four parameter model may be generated to model the building's energy consumption. Further energy consumption models may also be constructed in a similar manner based on their profiles, such as the one shown in illustration 200.

Referring now to FIG. 3, a block diagram of a processing circuit 300 configured to model and analyze a building's energy consumption is shown, according to an exemplary embodiment. In various embodiments, processing circuit 300 may be a component of a data acquisition and analysis service (e.g., data acquisition and analysis service 110 in FIG. 1) or any other computing device configured to analyze energy-related characteristics and statistics of a building.

Processing circuit 300 includes processor 302 and memory 304. Processor 302 may be or include one or more microprocessors (e.g., CPUs, GPUs, etc.), an application specific integrated circuit (ASIC), a circuit containing one or more processing components, a group of distributed processing components (e.g., processing components in communication via a data network or bus), circuitry for supporting a microprocessor, or other hardware configured for processing data. Processor 302 is also configured to execute computer code stored in memory 304 to complete and facilitate the activities described herein. Memory 304 can be any volatile or non-volatile computer-readable storage medium, or combinations of storage media, capable of storing data or computer code relating to the activities described herein. For example, memory 304 is shown to include computer code modules such as an energy consumption modeler 312, a fault detector 314, an energy score analyzer 316, and a report generator 318. When executed by processor 302, processing circuit 300 is configured to complete the activities described herein.

Processing circuit 300 also includes a hardware interface 306 for supporting the execution of the computer code energy consumption modeler 312, fault detector 314, energy score analyzer 316, and report generator 318. Interface 306 may include hardware configured to receive data as input to processing circuit 300 and/or communicate data as output to another computing device. For example, processing circuit 300 may receive building data 308 from one or more sensors, databases, or remote computing devices. Interface 306 may include circuitry to communicate data via any number of types of networks or other data communication channels. For example, interface 306 may include circuitry to receive and transmit data via a wireless network or via a wired network connection. In another example, interface 306 may include circuitry configured to receive or transmit data via a communications bus with other electronic devices.

Memory 304 may include building data 308. In general, building data 308 may include any data relating to the characteristics of one or more buildings. In some embodiments, building data 308 may include billing data from one or more utilities that supply the building with a consumable resource. For example, building data 308 may include billing data from a utility that provides the building with electrical power. In another example, building data 308 may include billing data from a utility that supplies water to the building.

Building data 308 may also include data regarding the physical characteristics of a building. For example, building data 308 may include data regarding the building's geographic location (e.g., street address, city, coordinates, etc.), dimensions (e.g., floor space, stories, etc.), use type (e.g., office space, hospital, school, etc.), or building materials. In some embodiments, these types of building data may be used by processing circuit 300 to allow a particular building energy consumption and other parameters to be compared to other buildings. For example, the building's modeled energy consumption may be normalized using the building's internal volume or area (e.g., the building's normalized energy consumption may be measured in kWh/ft²).

Memory 304 may also include weather data 310 which includes historical weather data for one or more geographic locations. Weather data 310 may include, for example, historical data regarding a location's temperature, humidity, atmospheric pressure, wind speed, precipitable water, or other weather-related data. In some embodiments, weather data 310 may be gathered via sensors located at or near a building under study. Weather data 310 may also include TMY data (e.g., TMY2, data, TMY3 data, etc.), according to various embodiments. Weather data 310 may also include weather data from any number of different time periods. For example, weather data 310 may include weather data down to the monthly, weekly, daily, or hourly level.

In some embodiments, memory 304 includes energy consumption modeler 312 configured to model the energy consumption of a building using building data 308 and weather data 310. Any form of model may be used by energy consumption modeler 312 to model a building's energy consumption. For example, energy consumption modeler 312 may use parametric models (linear regression, non-linear regression, etc.), nonparametric models (neural networks, kernel estimation, hierarchical Bayesian, etc.), or something in between, such as a Gaussian process model to model a building's energy consumption, according to various embodiments. In one embodiment, energy consumption modeler 312 models the energy consumption (E) of a building using linear regression as follows:

E=β₀+β₁x₁+ . . . +βₙxₙ+ε

where E is the dependent variable representing the energy consumption (e.g., measured in kilowatt-hours), xᵢ is an independent variable, βᵢ is an element of the parameter vector, and ε is an error factor (e.g., a noise factor). In other words, any number of independent variables may be used by energy consumption modeler 312 (e.g., weather data, occupancy data, etc.) within an energy consumption model to model a building's energy consumption. For example, energy consumption modeler 312 may model a building's energy consumption using a three parameter model (e.g., if only heating or cooling is used in the building), a four parameter model (e.g., if the building's heating and cooling balance points are equal), a five parameter model (e.g., if the building's heating and cooling balance points differ), or a regression model that uses other parameters.

Energy consumption modeler 312 may use any number of different estimation techniques to estimate the values of the model's coefficients (βᵢ) used in a building's energy consumption model. In some embodiments, energy consumption modeler 312 may use a partial least squares regression (PLSR) method to determine the parameter vectors. In further embodiments, energy consumption modeler 312 may use other methods, such as ridge regression (RR), principal component regression (PCR), weighted least squares regression (WLSR), or ordinary least squares regression (OLSR). Generally, a least squares estimation problem can be stated as follows: given a linear model

Y=Xβ+ε, ε˜N(0,σ²I)

find the vector \hat{β} that minimizes the sum of squared error RSS:

RSS=∥Y−X\hat{β}∥².

In the above equations, Y is a vector that contains the individual n observations of the dependent variable and X is a n by p+1 matrix that contains a column of ones and the p predictor variables at which the observation of the dependent variable was made. ε is a normally distributed random vector with zero mean and uncorrelated elements. According to various exemplary embodiments, other methods than using PLSR may be used (e.g., weighted linear regression, regression through the origin, etc.).

The optimal value of \hat{β} based on a least squares estimation has the solution:

\hat{β}=(X^{T}X)⁻¹X^{T}Y

where \hat{β} is a normal random vector distributed as:

\hat{β}˜N(β,σ²(X^{T}X)⁻¹).

The resulting sum of squared error divided by sigma squared is a chi-square distribution:

$$\frac{RSS}{{σ}^{2}} \sim {χ}_{n - ( p + 1 )}^{2} .$$

The difference in coefficients is distributed as:

Δβ=\hat{β}₁−\hat{β}₂˜N(0,σ²[(X₁^{T}X₁)⁻¹+(X₂^{T}X₂)⁻¹]).

The quadratic form of a normally distributed random vector where the symmetric matrix defining the quadratic form is given by the inverse of the covariance matrix of the normal random vector is itself a chi-square distributed random variable with degrees of freedom equal to the length of Δβ:

$$\frac{Δ {{β}^{T} [ {( {X}_{1}^{T} {X}_{1} )}^{- 1} + {( {X}_{2}^{T} {X}_{2} )}^{- 1} ]}^{- 1} Δ β}{{σ}^{2}} \sim {χ}_{p + 1}^{2} .$$

Additionally, the sum of two independent chi-square distributions is itself a chi-square distribution with degrees of freedom equal to the sum of the degrees of freedom of the two original chi-square distributions. Thus, the sum of the two root sum squared errors divided by the original variance is chi-square distributed, as:

$$\frac{{RSS}_{1} + {RSS}_{2}}{{σ}^{2}} \sim {χ}_{{n}_{1} + {n}_{2} - 2 ( p + 1 )}^{2} .$$

n₁ and n₂ are the number of data points used to estimate the model coefficients \hat{β}₁,\hat{β}₂.

According to various embodiments, energy consumption modeler 312 may normalize values relating to a building's energy consumption model. In some embodiments, energy consumption modeler 312 may normalize a building's energy consumption using the building's internal volume or area. For example, energy consumption modeler 312 may divide the building's utility data by the building's floor space to generate a normalized energy consumption value (e.g., measured in kWh/ft²).

In some embodiments, energy consumption modeler 312 may also use weather data 310 to normalize the modeled energy consumption of a building. Energy consumption modeler 312 may normalize a building's energy consumption by driving the building's energy consumption model using certain weather data, such as TMY data, to account for weather changes at a building's location. For example, a building's energy consumption may be higher in the summer than in the spring due to additional energy needed to cool the building. A cooling or heating degree day value may also be used by energy consumption modeler 312 to drive a building's energy consumption model. Generally, cooling degree days are calculated by integrating the positive difference between the time varying outdoor air temperature and the building's cooling breakeven temperature. Similarly, heating degree days are calculated by integrating the positive difference between the heating breakeven temperature and the time varying outdoor air temperature. Breakeven temperature corresponds to a single outdoor air temperature that coincides with the onset of the need for mechanical heating or cooling within the building. The integration interval is typically one month but other intervals may be used. For example, a cooling degree day (CDD) may be calculated as follows:

$$CDD = {\int }^{month} Max \{ 0 , ( {T}_{OA} - {T}_{BE} ) \} d t$$

where T_{OA} is the outdoor air temperature of the building and T_{BE} is the cooling breakeven temperature as previously defined. An alternative for calculating cooling or heating degree days is to assume a breakeven temperature (e.g. cooling breakeven temperature of 65° F.) regardless of the building characteristics. This approach is commonly used where breakeven temperatures are calculated based on geographical location (e.g. by city) in lieu of actual building characteristics. This approach is less accurate for building modeling but is common. Degree days may be used in the linear regression model by energy consumption modeler 312 as a dependent variable (e.g., as x₁). Degree days can also be used as statistics for benchmarking.

Energy consumption modeler 312 may store any resulting model coefficients, outputs, statistics, or other data related to a building's energy consumption model as model data 320. For example, model data 320 may include the determined model parameters (βᵢ), energy consumption (E), and any associated error measurements, such as a calculated RSS or coefficient of variation of a root mean square deviation (CVRMSE) score. In some embodiments, energy consumption modeler 312 may be further configured to generate and store data relating building parameters and energy consumption model parameters. For example, techniques for relating changes to model parameters and changes to building parameters are disclosed in U.S. patent application Ser. No. 13/759,933 entitled “SYSTEMS AND METHODS FOR EVALUATING A FAULT CONDITION IN A BUILDING,” filed by the same inventors of the present application on Feb. 5, 2013, the entirety of which is incorporated by reference herein. Energy consumption modeler 312 may also classify the data stored in model data 320 based on the buildings' classifications. For example, energy consumption modeler 312 may generate probability distribution functions using the model data of buildings having a certain usage type (e.g., hospitals, data centers, etc.) or geographic location (e.g., buildings located in temperate climates, moderate climates, etc).

Energy consumption modeler 312 may generate model data 320 for a particular building across multiple time periods. In one embodiment, energy consumption modeler 312 may use weather data 310 and building data 308 associated with a sliding temporal window to generate model data 320. For example, assume that building data 308 and weather data 310 are stored down to the monthly level. In such a case, one window may be a yearly window beginning with the month of August and ending with the month of July for the following year. A second window may then begin with the month of September and end with the month of August for the following year. In other embodiments, the windows may be shifted by time periods greater or smaller than one month. For example, a first window may begin on the first week of August, a second window may begin on the second week of August, etc.

According to various embodiments, memory 304 includes fault detector 314 which is configured to analyze model data 320 to detect a potential fault condition in a building. In some embodiments, fault detector 314 may analyze model data 320 from different temporal windows to detect a non-routine change to a building's energy consumption or its model's parameters. In such a case, a non-routine change to the building's energy consumption may be caused by an equipment fault. In some embodiments, fault detector 314 is also configured to diagnose a particular fault condition, in addition to determining whether a fault exists. For example, if fault detector 314 detects a non-route change to a building's energy consumption, it may also diagnose why the building's energy consumption has changed. In one embodiment, fault detector 314 may use a mapping between changes to building parameters and model parameters stored in model data 320 to diagnose a potential fault. For example, a change to a coefficient in the building's energy consumption model (e.g., ago may be mapped to one or more corresponding building parameters

Fault detector 314 may use any number of different analytical or statistical techniques to detect a potential fault. In one embodiment, fault detector 314 may generate a statistical process control chart to define operational limits for the values in model data 320. Such a control chart may be, but is not limited to, an exponentially-weighted moving average (EWMA) control chart, a cumulative sum (CUSUM) control chart, a Shewhart control chart, an Xbar chart, or any other form of statistical process control chart. The control chart generated by fault detector 314 may be trained using normalized consumption values in model data 320 from different time periods (e.g., data from a sliding timeframe). The limits of the resulting control chart may then be compared to data from a subsequent time frame in model data 320, to determine whether non-routine change has occurred.

In another embodiment, fault detector 314 calculates confidence intervals for a point estimate that corresponds with a new observation. For example, the new observation may be new utility billing data for a building in the most recent time period. Assuming that the independent and dependent variables of the building's energy consumption model do not contain measurement errors, only uncertainty in the model's regression coefficients may remain. Assuming also that the independent variables of the building's model are uncorrelated, fault detector 314 may use a confidence interval to determine whether the new observation falls outside of the confidence interval. If so, the new observation may be deemed a non-routine change and flagged by fault detector 314 as being a potential fault. In some cases, fault detector 314 may also generate a statistical measure that represents the probability of falsely identifying the new observation as being a non-routine change. For example, the confidence interval may be constructed such that a the new observation has a 5-10% probability of being falsely identified as being a non-routine change.

In another embodiment, fault detector 314 may utilize hypothesis testing to detect a non-routine change to the parameters of a building's energy consumption model. Fault detector 314 may determine a difference of multivariate measures of the change in model coefficients between two adjacent time periods to detect a non-routine change. For example, fault detector 314 may utilize the hypothesis testing techniques outlined in U.S. patent application Ser. No. 13/023,392, entitled “SYSTEMS AND METHODS FOR MEASURING AND VERIFYING ENERGY SAVINGS IN BUILDINGS,” filed by the same inventors of the present application on Feb. 8, 2011, which is hereby incorporated by reference in its entirety. Such a hypothesis test may test whether a null hypothesis corresponding to a routine change is valid. If the null hypothesis is rejected by fault detector 314, then a non-routine change has been detected and fault detector 314 may provide an indication that a potential fault exists.

In yet another embodiment, fault detector 314 may analyze model data 320 for a building to determine and analyze its recursive residuals. For example, assume that \hat{b}ᵣ, is the first r-number of OLSR estimates of the building's energy use model coefficients \hat{β} with k-number of independent variables. In one embodiment, fault detector 314 may calculate the recursive residual (wᵣ) corresponding to r as follows:

$${w}_{r} = \frac{{y}_{r} - {x}_{r}^{T} {b}_{r - 1}}{\sqrt{( 1 + {{x}_{r - 1}^{T} ( {X}_{r - 1}^{T} {X}_{r - 1} )}^{- 1} {x}_{r} )}}$$

where r=k+1, . . . , T, yᵣ is the rth observation (e.g., from the building's utility billing data), Xᵣ₋₁^{T}=[x₁, . . . , xᵣ₋₁], bᵣ=(Xᵣ^{T}Xᵣ)⁻¹Xᵣ^{T}Yᵣ, and Yᵣ^{T}=[y₁, . . . , yᵣ]. In some embodiments, fault detector 314 may utilize a CUSUM control chart to identify gradual shifts in the expected value of the recursive residual (wᵣ). In another embodiment, fault detector 314 may use a CUSUM of Squares test to detect idiosyncratic changes in the coefficients of the energy consumption model of the building. In a further embodiment, fault detector 314 may use EWMA control charts to detect a gradual shift in the expected value of the recursive residual. If fault detector 314 detects a shift in the recursive residual value, it may determine that a fault condition exists.

Memory 304 may include energy score analyzer 316 configured to determine the impact of a change to a building's systems on an energy score of the building, such as an Energy Star score. A change to the building's systems may be an implementation of an ECM, a repair to an equipment fault, an upgrade to a piece of equipment in the building, or another event that affects the building's energy consumption. In some embodiments, energy score analyzer 316 may use specific codes for types of equipment repairs, improvements, or ECMs stored in memory 304. For example, an upgrade to the building's lighting may have a different code than an upgrade to the building's air handling unit. Energy score analyzer 316 may use the stored code to determine changes to the building's energy score predicted to result from the corresponding act. For example, energy score analyzer 316 may determine the impact of a particular type of ECM on the building's Energy Star score, should the ECM be implemented.

Memory 304 may include report generator 318 configured to generate a report using data from fault detector 314 or energy score analyzer 316. A report generated by report generator 318 may be, but is not limited to, graphs (e.g., bar graphs, box and whisker graphs, etc.), tables, textual reports, and other forms of graphical representations. In one embodiment, report generator 318 may generate a report using data received from energy score analyzer 316 to convey potential changes to a building's energy score, should a particular event occur (e.g., implementing a particular ECM, correcting a fault condition, etc.). In another embodiment, report generator 318 may generate a report using data received from fault detector 314 to alert a user to a potential equipment fault.

Report generator 318 may provide a generated report to an electronic display directly or indirectly via interface 306. For example, report generator 318 may provide a generated report directly to an electronic display connected to interface 306. In another example, report generator 318 may provide a generated report to a remote device for display on the device's display (e.g., the report may be provided to a remote device connected to processing circuit 300 via a network). In a further example, report generator 318 may provide a generated report to a printer via interface 306.

In some cases, a report generated by report generator 318 may be used to set realistic priorities and goals when implementing energy conservation measures (ECMs) (e.g., by upgrading a building's HVAC equipment to more energy-efficient equipment). For example, assume that a report generated by report generator 318 indicates that a particular equipment upgrade will improve the building's Energy Star score by a certain amount. In such a case, the building's operator may evaluate different measures to prioritize or assess the effects of the measures.

In further cases, a report generated by report generator 318 may be used by an individual to identify potential equipment faults. For example, a building that has already implemented ECMs and has an energy consumption that is statistically higher than expected may be identified by fault detector 314. In such a case, a corresponding report by report generator 318 may identify the presence of a fault condition. In further embodiments, fault detector 314 is also configured to diagnose the cause of the fault condition and the generated report may identify the cause or potential causes of the fault.

### Fault Detection Using Model Data

Referring now to FIG. 4, a flow chart of a process 400 for identifying an equipment fault in a building is shown, according to an exemplary embodiment. Process 400 may be implemented by one or more computing devices, such as by a data acquisition and analysis service, by a building's control system, or the like. According to various embodiments, process 400 may be implemented by processing circuit 300 shown in FIG. 3. In general, process 400 allows for a non-routine change to a building's energy consumption model to be detected. Such a non-routine change may be attributable, for example, to an equipment fault in the building.

Process 400 includes generating an energy consumption model using readily-available building data (step 402). As used herein, readily-available building data refers to any building data that may be obtained without conducting an expensive energy audit or by deploying sensors throughout the building to monitor every aspect of the building's operation. Readily-available building data may be, for example, billing data from a utility (e.g., monthly billing data from an electric utility), weather data for the building (e.g., TMY data, etc.), or dimensional data regarding the building (e.g., the building's floor space, internal volume, etc.).

According to various embodiments, the generated energy consumption model is a regression model of the form:

E=β₀+β₁x₁+ . . . +βₙxₙ+ε

where E is the dependent variable representing the building's energy consumption (e.g., measured in kilowatt-hours), xᵢ is an independent variable, βᵢ is a model coefficient, and ε is an error factor (e.g., a noise factor). Regression models having different numbers of parameters may also be used, depending on the characteristics of the building under study (e.g., a three, four, five, etc., parameter regression model may be used). For example, a five parameter model may be used to model a building having separate cooling and heating balance points. The coefficients of the model (e.g., the βᵢ values) may be solved for using any number of different techniques, such as a OLSR, WLSR, PLSR, etc.

Process 400 also includes normalizing the consumptions from the building's energy consumption model (step 404). In some embodiments, the building's energy consumption may be divided by the building's internal area, to provide a normalized energy consumption per area value. In further embodiments, the building's model data may also be normalized to account for variations in the weather. Once an energy consumption model's coefficients have been determined, for example, the model may be driven using TMY or similar weather data to generate normalize consumption values. For example, a normalized annual consumption (NAC) value may be calculated by first generating the following regression model:

Y_{bill}=X_{Toa}β+ε with ε˜N(0,σ²I) and Y_{bill}˜N(X_{Toa}β,σ²I)

where Y_{bill} is billing data from a utility indicative of the building's energy consumption during a certain time window and X_{Toa} is a model parameter based on the building's outdoor air temperature. For example, the building's outdoor air temperature and a break even temperature may be used to determine a CDD or HDD value that may be used for X_{Toa}. The model's coefficients may then be determined by solving the following:

b=(X_{Toa}^{T}X_{Toa})⁻¹X_{Toa}^{T}Y

such that the following condition is minimized:

∥Y_{bill}−X_{Toa}b∥²

A NAC value may then be calculated as follows:

NAC=X_{TMY}b

where X_{TMY} is the model parameter corresponding to X_{TOA} but driven using TMY data. As a result, the energy consumption of the building is normalized to account for weather variations over the course of time.

Process 400 may include a decision point at which the time window used to generate the building's energy consumption model may be shifted (step 406). In some embodiments, the building's energy consumption model may be generated using building data from a sliding timeframe. For example, a first timeframe may include data ranging from January 2014 to December 2014 and a second timeframe may include data ranging from February 2014 to January 2105. In various embodiments, any number of different amounts of time may be used for the timeframe of the window and for the increments of time used to shift the time window. For example, a building's energy consumption model may be regenerated on a weekly basis by shifting the time window in weekly increments. In other words, steps 402, 404 of process 400 may be repeated any number of times to generate normalized model data that corresponds to different time windows.

Process 400 includes storing the model data from the generated energy consumption models for a building in an electronic storage device (step 408). The model data may include, for example, the inputs, coefficients, and outputs of the energy consumption models. Where the model is regenerated using building data from different time windows, different sets of model data corresponding to the different time windows may be stored. For example, a first set of model coefficients (e.g., β_{i,1}) may be determined and stored using data from January 2014 to December 2014, a second set of model coefficients (e.g., β_{i,2}) may be determined and stored using data from February 2014 to January 2015, etc.

Process 400 includes analyzing the stored model data to detect a non-routine change (step 410). In some embodiments, the independent variable of a building's energy consumption models (e.g., NAC or utility billing data) may be analyzed along the sliding time window, to detect a potential fault condition. For example, a statistical process control chart may be generated using the independent variables as training data. Newer independent variables can then be compared to the control limits of the control chart, to determine whether or not they fall outside of the control limits. In another example, a confidence interval may be constructed using the independent variables of the building's energy consumption models. If a new observation (e.g., a new NAC or utility billing data) falls outside of the confidence interval, this may indicate a non-routine change to the building's operations.

According to some embodiments, model coefficients for the building's energy consumption models may be used to detect a potential fault. In one embodiment, hypothesis testing may be used on the model coefficients to compare coefficients calculated across different time windows. For example, a hypothesis test may test a null hypothesis that a change in the model coefficients over time is routine. If this hypothesis is rejected, then a non-routine change has occurred. In a further embodiment, recursive residuals may be calculated using the model data from different time windows. Tests such as control charts, CUSUM, and CUSUM of Squares may then be applied to the recursive residuals, to detect non-routine changes in the energy consumption models.

Process 400 includes providing an indication of a potential fault condition (step 412). In some cases, a non-routine change in the model data across different time windows may indicate that a fault condition exists. In one embodiment, the indication may be provided to a fault diagnostic module, to determine the root cause of the potential fault. In another embodiment, the indication of the fault condition may be provided to an electronic display or as part of a printed report. For example, a user may be able to view a report that shows when a non-routine change to the building's energy consumption occurred.

Referring now to FIG. 5, a flow chart of a process 500 for using a control chart to identify an equipment fault in a building is shown, according to an exemplary embodiment. Process 500 may be implemented by any number of different computing devices, such as by a data acquisition service or processing circuit 300 shown in FIG. 3. In some embodiments, process 500 may be implemented in conjunction with another process, such as process 400, to identify the existence of a potential fault condition in a building. For example, process 500 may be implemented to perform step 410 of process 400. In general, process 500 utilizes a statistical process control chart to identify a non-routine change in an independent variable used in a building's energy consumption model.

Process 500 includes receiving model data for energy consumption models (step 502). In various embodiments, the model data may correspond to model data over a time series (e.g., model data generated across a sliding time window). The model data may also include independent variables used in the models over the sliding time periods. For example, the model data may include NAC values calculated across different time periods of a sliding time window (e.g., monthly NAC data generated using energy consumption models).

Process 500 also includes training a statistical process control chart model (step 504). In various embodiments, the model data generated from energy consumption models may be used to train a statistical process control chart. Such charts typically utilize upper and lower control limits relative to a center line to define the statistical boundaries for the process. New data values that are outside of these boundaries indicate a deviation in the behavior of the process. In some cases, the charts may also contain one or more alarm thresholds that define separate alarm regions below the upper control limit and above the lower control limits. A processor utilizing such a chart may determine that a new data value is within or approaching an alarm region and generate an alert, initiate a diagnostic routine, or perform another action to move the new data values away from the alarm regions and back towards the center line. Although this disclosure variously mentions the term “chart,” many of the exemplary embodiments of the disclosure will operate without storing or displaying a graphical representation of a chart. In such embodiments, an information structure suitable for representing the data of a statistical process control chart may be created, maintained, updated, processed, and/or stored in memory. Description in this disclosure that relates to systems having statistical process control charts or processes acting on or with statistical process control charts is intended to encompass systems and methods that include or act on such suitable information structures.

The trained control chart may utilize any form statistical process control technique including, but not limited to, EWMA or other moving average control charting techniques, CUSUM control charting techniques, Shewhart control charting techniques, Xbar control charting techniques, or any other form of process control charting technique. In general, a control chart may be trained by using the received model data to calculate a target parameter. For example, a target parameter may be an NAC value determined using the model data from different time periods across a sliding window of time. In one embodiment, the target parameter is the statistical mean of the models' independent variables. In another embodiment, the median of the independent variables is used. In yet another embodiment, a moving average of the independent variables can be used as the target parameter (e.g., a moving average, a weighted moving average, etc.).

In addition to determining a target parameter for the statistical process control chart, control limits may also be determined for the chart. In various embodiments, the control limits may be based on estimators of scale of the model data. Estimators of scale generally provide a metric that describes how spread out the model data is relative to the target parameter. For example, a estimator of scale for a normally-distributed or nearly normally-distributed set of model data may be based on the data's standard deviation. Such an estimator of scale may be used to determine the control chart limits. For example, the threshold control chart limits may be calculated using: threshold=μ±K*σ where K is a constant, μ is the target parameter and σ is the estimator of scale.

In one embodiment, the target parameter for an EWMA chart may be calculated as follows:

zᵢ=λxᵢ+(1−λ)zᵢ₋₁

where zᵢ₋₁ and zᵢ represents successive observations (e.g., model data associated with a sliding timeframe), xᵢ is the observation, and λ is a weighting factor. In such a case, the control limits for the EWMA chart may be calculated as follows:

$$T \pm LS \sqrt{\frac{λ}{2 - λ} [ 1 - {( 1 - λ )}^{2 i} ]}$$

where T is the estimated long-term process mean, and S is the estimated long-term standard deviation.

Process 500 calculating a new statistic for comparison to the control chart limits (step 506). In various embodiments, the new statistic may be a new independent variable from a building's energy consumption model. For example, if monthly NAC values are used to train the statistical process control chart in step 504, the new statistic may correspond to a new NAC value calculated using data from the previous month (e.g., by sliding the time window to encompass data from the previous month). In other embodiments, the statistic compared to the control chart may be from any number of different time frames.

Process 500 includes comparing the statistic to the control chart model (step 508). Once a statistical control chart has been trained using historical data from energy consumption models, the new statistic may be compared to the chart to determine whether the statistic represents a non-routine change. For example, the new statistic may be compared to the control chart limits to determine whether the new statistic falls outside of the range defined by the limits. If it does, this may indicate a non-routine change in the building's energy consumption and, therefore, a potential fault condition exists. In such a case, an indication of the detected non-routine change may be provided to a fault detection module or as part of a report, such as in step 412 of process 400.

Referring now to FIG. 6, a flow chart of a process 600 for using a confidence interval to identify an equipment fault in a building is shown, according to an exemplary embodiment. Process 600 may be implemented by any number of different computing devices, such as by a data acquisition service or processing circuit 300 shown in FIG. 3. In some embodiments, process 600 may be implemented in conjunction with another process, such as process 400, to identify the existence of a potential fault condition in a building. For example, process 600 may be implemented to perform step 410 of process 400.

Process 600 may use a confidence interval to determine whether a non-routine change to an energy consumption model's independent variable has occurred. In general, a confidence interval represents a range of values surrounding a point estimate for a population of values. For example, a point estimate may correspond to the mean of a subset of a larger population of values. In such a case, a confidence interval surrounding the point estimate may represent the probability of the true mean of the total population falling within the confidence interval. For example, point estimates for the population mean and standard deviation obtained from the sample mean X̅ and standard deviation S are:

\hat{μ}= X̅ and \hat{σ}=S

The sampling distributions of X̅ and S can be used to understand the margin of error in the point estimates. A 100(1−α) % confidence interval on the population mean μ can be calculated from the sampling distribution of the sample mean:

$$\overline{X} - {t}_{α / 2 , n - 1} \cdot \frac{S}{\sqrt{n}} < μ < \overline{X} + {t}_{α / 2 , n - {1}^{\cdot }} \cdot \frac{S}{\sqrt{n}}$$

where n equals the number data points in the sample. Likewise a 100(1−α) % confidence interval on the population variance (σ²) can be calculated from the sampling distribution of the sample variance S² as follows:

$$\frac{( n - 1 ) {S}^{2}}{{χ}_{α / 2 , n - 1}^{2}} < {σ}^{2} < \frac{( n - 1 ) {S}^{2}}{{χ}_{1 - α / 2 , n - 1}^{2}}$$

where X² is a chi squared distribution. In another embodiment, less than the full population may be used by finding the values such that a fraction of α/2 is less than the threshold and a fraction of α/2 is greater than the threshold. For near normal sample data, point and interval estimates can be used to infer information about the population statistics. Point estimates use sample data to derive a single number that is the most plausible value of a population statistic.

Process 600 includes receiving a test observation (step 602). In various embodiments, the test observation may correspond to one or more independent variables used in a building's energy consumption model. For example, the test observation may be a new consumption value or NAC value that results from a new monthly utility bill being issued. In various embodiments, the new observation may be separated temporally from its closest observation by any length of time. For example, observations regarding a building's energy consumption may be made on a monthly basis.

Process 600 includes determining a point estimate corresponding with the test observation (step 604). In various embodiments, the point estimate may be a sample mean value or other form of point estimate using some or all of the model data from different time windows. For example, model data from a time window that includes the test observation may be used to determine the point estimate (e.g., model data including data from the previous month may be used to determine the point estimate).

Process 600 also includes determining a confidence interval for the new observation (step 606). In one embodiment, the confidence interval may be calculated as follows:

$${Y}_{new} {=}^{Δ} {\hat{Y}}_{new} \pm {( Var ( {\hat{Y}}_{new} ) + Var ( ɛ ) )}^{1 / 2} * {t}_{α / 2 , n - p}$$

where Y_{new} corresponds to the new observation and Ŷ_{new} is the point estimate calculated based on the new observation and Var is used to denote the square of the standard error. Based on this, the following relationships also hold true:

Ŷ_{new}=X_{new}b

where X_{new} is an independent variable for the new energy consumption model parameter corresponding to the new observation and b represents the coefficients of the model calculated using least squares regression as follows:

b=(X^{T}X)⁻¹X^{T}Y

Regarding the variances, it is also known that they have the following relationships:

Var(Ŷ_{new})=X_{new}^{T}Var(b)X_{new}

Var(b)=σ²(X^{T}X)⁻¹

Var(ε)=σ²

Thus, the confidence interval for Y_{new} may be alternatively represented as follows:

$${Y}_{new} {=}^{Δ} {\hat{Y}}_{new} b \pm {s ( 1 + {{X}_{new}^{T} ( {X}^{T} X )}^{- 1} {X}_{new} )}^{1 / 2} * {t}_{α / 2 , n - p}$$

where s² is an unbiased estimator of σ²=RSS/(n−p). The value of α may be selected such that the confidence interval gives a 100(1−α) % degree of confidence in the population statistic. For example, α may be selected to be 0.05 or 0.1 to generate 95% or 90% confidence intervals, respectively.

Process 600 also includes determining whether the test observation (e.g., Y_{new}) falls within the calculated confidence interval. Since the value of α represents the degree of confidence in the interval, it may also represent the probability of falsely identifying the test observation as being a non-routine change. For example, if α=0.05, the confidence interval represents a 95% probability that the population statistic falls within the range. However, there still remains a 5% probability that the statistic is outside of the range. Thus, the value of α may also represent the false positive rate when using a confidence interval to detect a potential fault.

Referring now to FIG. 7, a flow chart of a process 700 for using hypothesis testing to identify an equipment fault in a building is shown, according to one embodiment. Similar to processes 500, 600, process 700 may be implemented by any number of different computing devices, such as by a data acquisition service or processing circuit 300 shown in FIG. 3. Also similar to processes 500, 600, process 700 may be implemented in conjunction with another process, such as process 400, to identify the existence of a potential fault condition in a building. For example, process 700 may be implemented to perform step 410 of process 500.

Process 700 includes receiving energy consumption model data (step 702). In cases in which a regression model is used to model the a building's energy consumption, the resulting model data may include model coefficients that can be analyzed to detect a potential fault condition. For example, a building's energy use may be modeled as follows:

X\hat{β}+r=Y

where X is a matrix containing the model's independent variables, \hat{β} is a vector containing the model coefficients (e.g., β₀, β₁, etc.), r is the vector containing the residuals, and Y is a vector of estimated energy consumption values normalized by building floor area. A regression technique (e.g., OLSR, WLSR, etc.) may then be used to solve for the vector \hat{β} containing the regression model coefficients. For example, a least squares regression has the following solution for the model coefficients:

\hat{β}=(X^{T}X)⁻¹X^{T}Y

where X^{T} is the transpose of the matrix X.

According to various embodiments, the received model coefficients may be associated with different temporal windows (e.g., data used in the regression models to determine the coefficients may be from different time periods). When a building is operating in a consistent manner (e.g., consistent energy consumption) and the baseline model for the building includes all the independent predictor variables necessary to accurately estimate the energy consumption, the coefficients of the baseline model should remain constant over time. Therefore, if two temporally consecutive windows of data from time intervals [tₐ,t_{b}] and [t_{b},t_{C}] are used, the difference in two baseline model coefficients should be near zero. In various embodiments, the received model coefficients may correspond to data from temporally-adjacent windows or data from time intervals having a slight “gap” between the two intervals (e.g., some data points may be omitted between the time intervals). The difference in model coefficients can be represented as:

Δβ=\hat{β}₁−\hat{β}₂

where Δβ is the difference between the baseline model coefficients from window one and window two \hat{β}₁,\hat{β}₂, respectively. Because the baseline model coefficients have physical meaning (e.g., cost per cooling degree day), unexpected changes in coefficients over time can advantageously be linked to root causes (e.g., chiller fouling, decrease in setpoints, etc.).

Process 704 also includes generating a null-hypothesis for testing (step 704). For the received model coefficients, there may be random variation in a coefficient, the magnitude of which is based on, for example: the number of periods or data points in the time intervals, the variance of the errors of the baseline model, the number of predictor variables used in the model, and the values of the predictor variables during each of the two time intervals. Additionally, the values of the predictor variables during each time interval can have a significant effect of the variation of the coefficients. Thus, in one embodiment, hypothesis testing may be used to determine whether the difference in the coefficients is large enough to be considered statistically significant or whether the coefficient difference is due to the random variation described above, rather than a real change in a static factor affecting the building's energy use.

In various embodiments, the generated hypothesis may include a null hypothesis corresponding to a constant baseline model (e.g., the normalized energy consumptions of the building during the two time intervals remain constant). Null hypothesis testing generally tests whether a null hypothesis is to be rejected. In other words, two outcomes are possible: the null hypothesis is rejected or the null hypothesis fails to be rejected. A failure to reject the null hypothesis does not guarantee, however, the validity of the null hypothesis. In cases in which the null hypothesis corresponds to a constant baseline model, rejection of the null hypothesis may indicate that a non-routine change has occurred in the building's operation (e.g., that a fault condition may exist).

The null hypothesis may be represented by at least one test statistic related to the difference in a coefficient or a set of coefficients from the two data sets. If two consecutive windows of data are used to build similar baseline models (i.e., the coefficients of the models are similar) and static factor changes have not occurred during the time period of the windows, then the test statistic should be small (i.e., within the expected amount of random variation). In various embodiments, the test statistic is an F-statistic or a Z-statistic. For example, each vector of model coefficients may be a normal random vector distributed as follows:

\hat{β}˜N(β,σ⁻²(X^{T}X)⁻¹).

The resulting sum of squared error divided by sigma squared is a chi-square distribution:

$$\frac{RSS}{{σ}^{2}} \sim {χ}_{n - ( p + 1 )}^{2} .$$

The difference in the model coefficients is then distributed as follows:

Δβ=\hat{β}₁−\hat{β}₂˜N(0,σ²[(X₁^{T}X₁)⁻¹+(X₂^{T}X₂)⁻¹]).

The quadratic form of a normally distributed random vector where the symmetric matrix defining the quadratic form is given by the inverse of the covariance matrix of the normal random vector is itself a chi-square distributed random variable with degrees of freedom equal to the length of Δβ:

$$\frac{Δ {{β}^{T} [ {( {X}_{1}^{T} {X}_{1} )}^{- 1} + {( {X}_{2}^{T} {X}_{2} )}^{- 1} ]}^{- 1} Δ β}{{σ}^{2}} \sim {χ}_{p + 1}^{2} .$$

Additionally, the sum of two independent chi-square distributions is itself a chi-square distribution with degrees of freedom equal to the sum of the degrees of freedom of the two original chi-square distributions. Thus, the sum of the two sum of squared errors divided by the original variance is chi-square distributed, as:

$$\frac{{RSS}_{1} + {RSS}_{2}}{{σ}^{2}} \sim {χ}_{{n}_{1} + {n}_{2} - 2 ( p + 1 )}^{2} .$$

where n₁ and n₂ are the number of data points used to estimate the model coefficients \hat{β}₁,\hat{β}₂. Finally, the ratio of two chi-square distributions divided by their respective degrees of freedom is an F-distributed random variable:

$${F}_{Δβ} = ( \frac{{{Δβ}^{T} [ ( {X}_{1}^{T} {X}_{1} ) + {( {X}_{2}^{T} {X}_{2} )}^{- 1} ]}^{- 1} Δβ}{{RSS}_{1} + {RSS}_{2}} ) ( \frac{{n}_{1} + {n}_{2} - 2 ( p + 1 )}{p + 1} ) ∼ {F}_{p + 1 , {n}_{1} + {n}_{2} - 2 ( p + 1 )} .$$

F_{Δβ} is defined as the test statistic. As Δβ moves away from the origin, F_{Δβ} increases. Further, the maximum increase occurs in the direction of the least variance of the model coefficients and is scaled by the sum of squared errors. Thus, F_{Δβ} is based on changes in model coefficients which can easily be related back to a root cause and it takes into account the random variation of the changes of the model coefficients even when the model is stationary. The F_{Δβ} statistic may further be converted into a standard normal variable Z_{Δβ} by the proper transformation function.

Process 700 also includes determining whether the null-hypothesis is rejected (step 706). Once a test statistic has been determined, the test statistic may be compared to a critical value to determine whether the null hypothesis is rejected. For example, the resulting F_{Δβ} or Z_{Δβ} statistic from comparing model coefficients from different time intervals can be used as the test statistic for purposes of hypothesis testing. The null hypothesis is rejected if the F-statistic F_{Δβ} is greater than its critical value f_{crit} which may be calculated using F_{p+1,n}_{₁}₊ₙ_{₂}₋₂₍ₚ₊₁₎(1−α) where F⁻¹ is the inverse of the cumulative F-distribution with the required degrees of freedom. In other words, the null hypothesis is rejected and a static factor can be determined to have changed when F_{Δβ}>f_{crit}. In some embodiments, a user may determine an acceptable level for α, the probability of rejecting the null hypothesis when it is in fact valid. In some embodiments, an automated process uses α to determine the critical value for use in accepting or rejecting the null hypothesis.

According to some embodiments, process 700 may be repeated. For example, different data sets may be used to on a rolling basis (e.g., by shifting the time windows temporally) to assess the most recent building data. For example, new data points may be generated with new utility billing data, which may be received daily, weekly, monthly, or at any other periodic interval. With multiple hypothesis tests, however, correlation of the test statistics may largely impact the conservativeness of typical methods for suppressing the family-wise probability of falsely rejecting the null hypothesis (Bonferroni's method, for example). For example, if multiple statistics of two data sets are highly correlated, the statistics do not differ by a significant amount. Thus, direct applications of Bonferroni's method would be very conservative and greatly reduce the power of the test (probability of correctly identifying a change in the model coefficients).

In the embodiments of the present disclosure, if static factors are not changing, the statistics calculated using the windowing method described previously should be highly correlated. Window data selection steps described above could be designed to maintain this high correlation during normal behavior. For example, during the reporting period, the last data point inserted into the second data window replaces the oldest data point in the first data window, meaning that only two data points have changed since the last calculation. In one embodiment, an inverse cumulative distribution function (CDF) of the test statistics may be evaluated. Evaluation of the inverse CDF can be phrased as, given a value p (e.g., a desired probability), find a value x such that P(X<x)=p, where X is a random variable, in the current disclosure the maximum of the sequence of statistics and x is the argument of the CDF, which in the current disclosure corresponds with the critical value of the null hypothesis. In context of the present disclosure, this means the inverse CDF is used to determine a critical value such that the probability that the maximum of the sequence of statistics is equal to the desired probability p typically equal to one minus the indicated probability of falsely rejecting the null hypothesis.

If several samples are drawn from the distribution of data points, a point estimate for the probability p is given by:

$$\hat{P} ( X < x ) = \hat{p} = \frac{{n}_{\{ X < x \}}}{n}$$

with an associated 1−a confidence interval for p. The confidence interval 1−a indicates a desired probability that the true value of p resides within the band \hat{p} plus or minus the tolerance. A desired probability p (e.g., the p value of P(X<x)=p) and a confidence interval for p may then be determined. In one embodiment, the desired probability p and confidence interval may be chosen by a user. The confidence interval should be determined such that probabilities with values on the upper and lower limits of the interval are accepted at the 1−a confidence level. In such a case, a value may be returned such that all probabilities within the 1−a confidence interval are included in the range defined by the upper and lower limits. This guarantees that the probability that the actual value of p for the returned value is between the upper and lower limits is greater than 1−a.

In some embodiments, the number of samples required to draw from the distribution in order to reach a desired tolerance may also be determined. The number of samples n may be found by using an iterative root finding technique where the objective is to find n such that:

$$max ( \begin{matrix}\frac{n \hat{p} {F}_{2 n \hat{p} , 2 [ n ( 1 - \hat{p} ) + 1 ]}^{- 1} ( \frac{a}{2} )}{n ( 1 - \hat{p} ) + 1 + n \hat{p} {F}_{2 n \hat{p} , 2 [ n ( 1 - \hat{p} ) + 1 ]}^{- 1} ( \frac{a}{2} )} - low limit , \\ high limit - \frac{( n \hat{p} + 1 ) {F}_{2 [ n \hat{p} + 1 ] , 2 n ( 1 - \hat{p} )}^{- 1} ( 1 - \frac{a}{2} )}{n ( 1 - \hat{p} ) + ( n \hat{p} + 1 ) {F}_{2 [ n \hat{p} + 1 ] , 2 n ( 1 - \hat{p} )}^{- 1} ( 1 - \frac{a}{2} )}\end{matrix} ) = 0 ,$$

where \hat{p} is given the value of p and the low limit and high limit are the upper and lower limits of the 1−a confidence interval.

A certain number of samples (n) of the distribution may be drawn at random. For example, the samples can be drawn by simulating a linear model and performing the process in order to do an approximation from a multivariate normal distribution. Using the samples, a critical value x is found such that the total number of samples n drawn less than x is equal to np (e.g., the number of samples times the probability of each individual sample being less than x) and the total number of samples greater than x is equal to n(1−p) (e.g., the number of samples times the probability of each individual sample being greater than X).

The 1−a confidence interval for p may also be recalculated. The equation used for the calculation may be the following:

$$P ( \frac{n \hat{p} {F}_{2 n \hat{p} , 2 [ n ( 1 - \hat{p} ) + 1 ]}^{- 1} ( \frac{a}{2} )}{n ( 1 - \hat{p} ) + 1 + n \hat{p} {F}_{2 n \hat{p} , 2 [ n ( 1 - \hat{p} ) + 1 ]}^{- 1} ( \frac{a}{2} )} < p < \frac{( n \hat{p} + 1 ) {F}_{2 [ n \hat{p} + 1 ] , 2 n ( 1 - \hat{p} )}^{- 1} ( 1 - \frac{a}{2} )}{n ( 1 - \hat{p} ) + ( n \hat{p} + 1 ) {F}_{2 [ n \hat{p} + 1 ] , 2 n ( 1 - \hat{p} )}^{- 1} ( 1 - \frac{a}{2} )} ) = 1 - a .$$

The critical value is found by taking the smallest value that will result in a fraction of samples less than x to be greater than p. The value of x may then be used to detect non-routine changes in the model coefficients by evaluating the null hypothesis of a constant baseline. For example, x may be used as the critical value f_{crit} and compared to the F-statistic F_{Δβ} to evaluate the null hypothesis. If F_{Δβ}>f_{crit}, the null hypothesis is rejected and a non-routine change to the building

Referring now to FIG. 8, a flow chart of a process 800 for using recursive residuals to identify an equipment fault in a building is shown, according to an exemplary embodiment. Process 800 may be implemented by any number of different computing devices, such as by a data acquisition service or processing circuit 300 shown in FIG. 3. In some embodiments, process 800 may be implemented in conjunction with another process, such as process 400, to identify the existence of a potential fault condition in a building. For example, process 800 may be implemented to perform step 410 of process 400.

In general, recursive residuals may be used to test the constancy of regression relationships over time. For example, the energy consumption model of a building may be recalculated any number of times using a time series of building data. Similar to the null-hypothesis testing disclosed in process 700, a null hypothesis may correspond to the coefficients of an energy consumption model and their corresponding error variance being time-invariant. In process 800, such a null-hypothesis may be evaluated through the use of recursive residuals to test for non-routine changes in the energy consumption model parameters over time (e.g., by determine whether the hypothesis is rejected). For example, the recursive residuals may be calculated according to the techniques described in the article, “Techniques for Testing the Constancy of Regression Relationships over Time” by R. L. Brown, et. al., and published in the Journal of the Royal Statistical Society, Series B (Methodological), Vol. 37, No. 2 (1975), pp. 149-192, the entirety of which is hereby incorporated by reference.

Process 800 includes receiving data associated with an energy consumption model for a building (step 802). The received data may include, for example, model coefficients for a regression model. Similar to step 702 of process 700, the model coefficients (e.g., a vector \hat{β}) may be calculated using a regression technique (e.g., OLSR, WLSR, etc.). According to various embodiments, the received model coefficients may also be associated with different temporal windows (e.g., data used in the regression models to determine the coefficients may be from different time periods). In one embodiment, the windows may be temporally adjacent to one another (e.g., a second window beings immediately after a first window ends).

Process 800 also includes calculating recursive residuals using the received model data (step 804). In one embodiment, a recursive residual (wᵣ) may be calculated for the first r number of observations as follows:

$${w}_{r} = \frac{{y}_{r} - {x}_{r}^{′} {b}_{r - 1}}{\sqrt{( 1 + {{x}_{r}^{′} ( {X}_{r - 1}^{′} {X}_{r - 1} )}^{- 1} {x}_{r}}} ( r = k + 1 , … , T )$$

where k is the number of regressors used in the energy consumption model and bᵣ₋₁ is the least squares estimate of \hat{β} based on the first (r−1) number of observations (e.g., bᵣ₋₁=(Xᵣ₋₁′Xᵣ₋₁)⁻¹(Xᵣ₋₁′Yᵣ)), Xᵣ₋₁′=[x₁, . . . , xᵣ₋₁], and Yᵣ₋₁′=[y₁, . . . , yᵣ₋₁]. It should be noted that the numerator of the calculation is a modified residual using the most current values of the independent and dependent variables from the energy consumption model (e.g., xᵣ and yᵣ), while the model coefficients are time lagged from the previous time window (e.g., bᵣ₋₁ is used). The denominator, meanwhile, represents the amount of uncertainty of the model coefficients and their idiosyncratic errors, assuming that the independent and dependent variables used in the energy consumption model are error-free. Any number of different time periods can be selected for review, allowing for a corresponding number of recursive residuals to be calculated (e.g., by adjusting the value of T to generate T−(k+1) recursive residuals).

Process 800 also includes analyzing the calculated recursive residuals to detect a non-routine change in the building's operation (step 806). Once the recursive residuals have been calculated, the set of residuals may be analyzed to detect a shift in the set of residuals. For example, a shift in the mean of the recursive residuals may be detected as a potential equipment fault in the building. According to various embodiments, the calculated recursive residuals may then be tested using a CUSUM test (e.g., to detect a departure in the mean of the recursive residuals), a CUSUM of Squares test (e.g., to detect idiosyncratic changes in the model coefficients), or a control chart technique (e.g., to detect a shift in the expected value of the recursive residuals).

A control chart may be constructed to test the recursive residuals in a manner similar to those disclosed in process 500. The control chart may utilize any form of statistical process control such as, but not limited to, EWMA, CUSUM, Shewhart, or Xbar control techniques. In a preferred embodiment, an EWMA control chart is used, since EWMA charts are sensitive to gradual shifts in the recursive residuals. In general, a target parameter may first be generated using the set of recursive residuals. For example, the mean, EWMA, or other target parameter may be generated using the calculated recursive residuals. Similarly, the standard deviation, calculated EWMA control limits, or other values may be calculated using the target parameter and the recursive residuals to define limits around the target parameter. If the expected value of the recursive residuals shifts beyond the control limits, a non-routine shift has been detected and may correspond to a fault condition being present in the equipment of the building.

Energy Score Estimations Using Model Data

In addition to using an energy consumption model to detect potential faults in the building's equipment, a building's energy consumption model may also be used to evaluate the effects of potential changes to the building's systems. Potential changes to the building's systems may include, but are not limited to, EOMs and facility improvement measures (FIMs). According to various embodiments, the impact of implementing an ECM or FIM may be translated into an Energy Star score for the building, allowing the building's operator to evaluate the impact of different EOMs or FIMs.

To calculate an ENERGY STAR score, an Efficiency Ratio (ER) value must first be determined. In general, an ER value is the actual source EUI divided by the calculated source EUI obtained from a linear regression model. The regression model coefficients are provided by ENERGY STAR and different models are specified for different building types. For an office building, for example, the ENERGY STAR model has 6 inputs: ft², # PCs, weekly operating hours, worker density, HDD, and CDD. Accordingly, the ER value for the building can be determined as follows:

$${ER}_{office bldg} = \frac{actual source energy use intensity = f ( bill data , {ft}^{2} )}{\begin{matrix}predicted source energy use intensity = \\ f ( {ft}^{2} , \# PCs , operating hrs , {ρ}_{workers} , HDD , CDD )\end{matrix}}$$

To calculate a building's Energy Star score, a determined ER value for the building can be used as input to a two parameter cumulative gamma distribution. For example, the building's Energy Star score may be determined as follows:

Energy Star Score=Round(100*(1−gammaCDF(ER,5.646,0.1741)))

The resulting ENERGY STAR score (0-100%) reflects the percentage of similar buildings nationwide with higher source EUIs than the building under study.

Referring now to FIG. 9, a flow chart of a process 900 for using a building model to determine an Energy Star score is shown, according to an exemplary embodiment. Process 900 may be implemented by any number of different computing devices, such as by a data acquisition service or processing circuit 300 shown in FIG. 3. Process 900 allows for the potential impact on a building's Energy Star score to be evaluated, should a particular ECM, FIM, or other action that affects the building's energy consumption be implemented. In general, process 900 operates by first determining energy use intensity values for a base case (e.g., using historical values from the building's actual operation) and for an adjusted case (e.g., using predicted adjustments to the building's energy model coefficients as a result of implementing a FIM or ECM). These values may then be used to determine a predicted Energy Star score for the building based on the changes to the building's energy use intensities.

Process 900 includes receiving utility data (step 902). Utility data may include any information regarding the energy use or consumption by a building. The utility data may also be from any number of different timeframes. In one embodiment, the received utility data may include one year's worth of energy consumptions, broken down by month. For example, the received utility data may be the building's monthly energy consumptions in the previous year. The utility data may be received directly from the utility, from a meter or other sensor that measures energy consumption by the building, or from another source (e.g., a computer server that stores utility data for the building).

Process 900 includes receiving building data (step 904). Building data may generally include any measured value relating to the physical state of the building. In various embodiments, the building data includes readily-available information regarding the building, thereby allowing the building's energy consumption to be modeled in the lean manner disclosed herein. As shown, the received building data may include data regarding the physical dimensions of the building (e.g., the floor space of the building measured in ft²). Also as shown, the received building data may include data regarding the building's location (e.g., the building's street address, zip code, city, state, region, latitude and longitude, etc.).

Process 900 also includes determining weather-related data for the building (step 906). In various embodiments, measured weather data at or near the building may be used to determine parameters such as an outdoor air temperature (T_{OA}), CDD values, HDD values, or other weather-related parameters. For example, the building's zip code received in step 903 may be used to retrieve the outdoor air temperature for the building's zip code in the past twelve months. These temperature values may then be used to calculate CDD or HDD values, as discuss previously. In one embodiment, the weather data may also correspond to the same time interval as the building data received in step 902. For example, monthly CDD or HDD values may be determined for the previous year, if the utility data received in step 902 includes energy consumption data from the previous twelve months.

Process 900 also includes determining model parameters for a baseline energy consumption model (step 908). According to various embodiments, the utility data (e.g., the building's energy consumption over the previous twelve months) and corresponding degree day values (e.g., CDD and/or HDD values) may be used in an inverse regression model to determine baseline heating and cooling related model coefficients as follows:

β_{base,clg}=(X_{clg}^{T}X_{clg})⁻¹X_{clg}^{T}Y_{clg} and β_{base,htg}=(X_{htg}^{T}X_{htg})⁻¹X_{htg}^{T}Y_{htg}

with X_{clg}, X_{htg}, and Y being defined as follows:

$${X}_{clg} = [ \begin{matrix}1 & {η}_{days , 1} & {CDD}_{1} \\ … & … & … \\ 1 & {η}_{days , 12} & {CDD}_{12}\end{matrix} ] , {X}_{htg} = [ \begin{matrix}1 & {η}_{days , 1} & {HDD}_{1} \\ … & … & … \\ 1 & {η}_{days , 12} & {HDD}_{12}\end{matrix} ] , \\ and Y = \frac{1}{area} [ \begin{matrix}{kWh}_{1} \\ … \\ {kWh}_{12}\end{matrix} ]$$

where area (e.g., the building's floor space measured in ft²) is used to normalize the building's energy use model parameters. The coefficients of the baseline regression model (e.g., the vector β_{base}) may be calculated using a regression technique, such as OLSR, PLSR, WLSR, or any other technique to determine regression model coefficients. These coefficients are related to building parameters and operational settings, such as the building's overall cooling or heating equipment efficiency, outdoor ventilation rates, envelope conductance area products, zone temperature setpoints, and internal heat gains for the base case.

Process 900 includes determining adjusted model coefficients based on a received identifier for a type of change to the building (step 910). In some embodiments, a unique identifier may be used to represent different FIMs or other actions that may affect the building's energy consumption. For example, a particular identifier may correspond to replacing the lighting used in the building with energy-efficient bulbs, such as compact fluorescent light (CLF) bulbs or light emitting diode (LED) bulbs. In another example, the received identifier may correspond to adjusting the operation of the building's existing equipment, such as automatically dimming the lights in the building at nighttime or when the building's occupancy is minimal.

According to various embodiments, each action identifier may have associated changes to a building's energy consumption model coefficients (e.g., Δβs). An action identifier may have a corresponding change to the building's parameters, which may be mapped to changes in the model's coefficients. For example, a five parameter energy consumption model may be defined as follows:

E=β₀+β₁(T_{OA}−β₂)+β₃(β₄−T_{OA})+ε

where β₀ is the building's base energy consumption (E₀), β₁ is the building's cooling slope (S_{C}), β₂ is the building's cooling break even temperature (T_{bC}), β₃ is the building's heating slope (S_{H}), and β₄ is the building's heating break even temperature (T_{bH}). Thus, the coefficients in this energy consumption model may be represented by a five-dimensional vector as follows:

$${φ}_{M} = [ \begin{matrix}{E}_{0} \\ {T}_{bC} \\ {T}_{bH} \\ {S}_{c} \\ {S}_{H}\end{matrix} ] ∈ {R}^{5}$$

where E₀ is the building's base energy load, S_{H} is the building's heating slope, S_{C} is the building's cooling slope, T_{bH} is the building's heating break even temperature, and T_{bC} is the building's cooling break even temperature.

The energy consumption model coefficients are related to building parameters (e.g., physical parameters of the building) as follows:

$${C}_{C} = UA + {V}_{C} ρ {c}_{p}$$

$${C}_{H} = UA + {V}_{H} ρ {c}_{p}$$

$${S}_{C} = \frac{{C}_{C}}{{η}_{C}}$$

$${S}_{H} = \frac{{C}_{H}}{{η}_{H}}$$

$${T}_{bC} = {T}_{sp} - \frac{{Q}_{i}}{{C}_{C}}$$

$${T}_{bH} = {T}_{sp} - \frac{{Q}_{i}}{{C}_{H}}$$

where C_{c} is the building's cooling coefficient (e.g., measured in kW/day*° F.), C_{H} is the heating coefficient (e.g., measured in kW/day*° F.), U is the overall envelope conductance, A is the envelop area, V_{H} is the sum of heating ventilation and infiltration flow rate, V_{C} is the sum of cooling ventilation and infiltration flow rate, ρ is the density of air, cₚ is the specific heat of air, η_{C} is the cooling efficiency, η_{H} is the heating efficiency, T_{b,C} is the cooling break even temperature, T_{b,H} is the heating break even temperature, Tₛₚ is the setpoint temperature of the building's HVAC system, and Qᵢ is the internal building load (e.g., measured in kW/day). It is also assumed that the building's internal load (Qᵢ) is related to the building's base energy (E₀) plus a constant (c) as follows:

Qᵢ=E₀+c

where c is also measured in (kW/day).

For purposes of mapping building parameters to energy consumption model parameters, a ventilation coefficient (C_{V}) may be used to account for both infiltration through the envelope and a minimum forced ventilation. Similarly, an economizer coefficient (C_{E}) may be used to account for the maximum forced ventilation through the building's economizer that is part of the building's HVAC system. Using these two coefficients gives the following:

$${C}_{C} = UA + {V}_{min} ρ {c}_{p}$$

$${C}_{E} = ( {V}_{max} - {V}_{min} ) ρ {c}_{p}$$

$${S}_{C} = \frac{{C}_{V}}{{η}_{C}} \\ {S}_{H} = \frac{{C}_{V}}{{η}_{H}} \\ {T}_{bC} = {T}_{sp} - \frac{{Q}_{i}}{{C}_{V} + {C}_{E}} \\ {T}_{bH} = {T}_{sp} - \frac{{Q}_{i}}{{C}_{V}}$$

where Cᵥ is the ventilation coefficient and C_{E} is the economizer coefficient. Based on these equations, the building's parameters may be represented as a six dimensional vector as follows:

$${φ}_{B} = [ \begin{matrix}{C}_{V} \\ {C}_{E} \\ {T}_{sp} \\ {Q}_{i} \\ {η}_{C} \\ {η}_{H}\end{matrix} ] ∈ {R}^{6}$$

A projection matrix relating the building parameters and energy consumption model coefficients may also be determined. As described previously, a vector of building parameters may have more parameter values than a vector of energy consumption model coefficients. For example, a parameter vector for a five parameter energy consumption model may have a corresponding six dimensional building parameter vector. In one embodiment, assumptions may be made regarding some of the building parameters such that the remaining building parameters can be calculated. For example, it is possible to assume a temperature setpoint for the building (e.g., Tₛₚ=75° F.) and that its internal load is 50% greater than its base load (e.g., Qᵢ=1.5*E₀). These assumptions allow for the calculation of the remaining building parameters (e.g., C_{V}, C_{E}, η_{C}, and η_{H}).

According to various embodiments, sensitivity analysis may be used to determine how a change in the building parameters affects the model parameters or vice-versa. In one embodiment, changes to the building parameters may be related to changes in the model parameters as follows:

$$Δ {S}_{C} = ( \frac{1}{{η}_{C}} ) Δ {C}_{V} - ( \frac{{C}_{V}}{{η}_{C}^{2}} ) {Δη}_{C}$$

$$Δ {S}_{H} = ( \frac{1}{{η}_{H}} ) Δ {C}_{V} - ( \frac{{C}_{V}}{{η}_{H}^{2}} ) {Δη}_{H}$$

$${T}_{bC} = Δ {T}_{sp} - ( \frac{1}{{C}_{V} + {C}_{E}} ) Δ {Q}_{i} + ( \frac{{Q}_{i}}{{( {C}_{V} + {C}_{E} )}^{2}} ) ( Δ {C}_{V} + Δ {C}_{E} )$$

$${T}_{bH} = Δ {T}_{sp} - ( \frac{1}{{C}_{V}} ) Δ {Q}_{i} + ( \frac{{Q}_{i}}{{C}_{V}^{2}} ) Δ {C}_{V}$$

$$Δ {E}_{0} = Δ {Q}_{i}$$

These equations may alternatively be represented in matrix form as follows:

$$[ \begin{matrix}Δ {E}_{0} \\ Δ {T}_{bc} \\ Δ {T}_{bH} \\ Δ {S}_{C} \\ Δ {S}_{H}\end{matrix} ] = [ \begin{matrix}0 & 0 & 0 & 1 & 0 & 0 \\ ( \frac{{Q}_{i}}{{( {C}_{V} + {C}_{E} )}^{2}} ) & ( \frac{{Q}_{i}}{{( {C}_{V} + {C}_{E} )}^{2}} ) & 1 & ( \frac{- 1}{{C}_{V} + {C}_{E}} ) & 0 & 0 \\ ( \frac{{Q}_{i}}{{C}_{V}^{2}} ) & 0 & 1 & ( \frac{- 1}{{C}_{V}} ) & 0 & 0 \\ ( \frac{1}{{η}_{C}} ) & 0 & 0 & 0 & ( \frac{- {C}_{V}}{{η}_{C}^{2}} ) & 0 \\ ( \frac{1}{{η}_{H}} ) & 0 & 0 & 0 & 0 & ( \frac{- {C}_{V}}{{η}_{H}^{2}} )\end{matrix} ] [ \begin{matrix}Δ {C}_{V} \\ Δ {C}_{E} \\ Δ {T}_{sp} \\ Δ {Q}_{i} \\ Δ {η}_{C} \\ Δ {η}_{H}\end{matrix} ]$$

The above equation gives rise to a matrix (A) as follows:

Δβ=Δφ_{M}=AΔφ_{B}

where A is a matrix that maps building parameter changes to model parameter changes and vice-versa. Thus, a known change a building's physical parameters that would result from a particular action may be mapped to changes in the building's energy consumption model coefficients. For example, an upgrade to a building's economizer may affect the building's economizer coefficient and, correspondingly, the coefficients of the building's energy consumption model.

Process 900 also includes receiving a site to source energy conversion value (step 912). In general, energy may be classified as being either primary energy or secondary energy. Primary energy represents the electrical or thermal energy obtained on site using raw fuel (e.g., natural gas, fuel oil, etc.). For example, a building may have a furnace that burns natural gas to provide internal heating to the building. Secondary energy, in contrast, refers to the electrical or thermal energy received directly by the building. For example, the building may receive electrical energy directly from a grid or thermal energy from a municipal steam system. To assess a building's efficiency, such as when an Energy Star score is determined, a site to source conversion value may be used to convert primary and secondary energy into equivalent source energy values. In some cases, a national or regional average may be used for the conversion value. For example, the U.S. EPA uses a site to source conversion of 1 kWhₛᵢₜₑ=3.34 kWh_{source} for electricity consumption, which is the national average of conversion values between the years 2001 and 2005. Other site to source energy conversion values may be used, as promulgated by the U.S. EPA and can be obtained at the following url: http://www.energystar.gov/ia/business/evaluate_performance/site_source.pdf?5397-ce1d.

Process 900 also includes receiving historical weather data (step 914). The received historical weather data may include TMY data, such as TMY2 data, TMY3 data, etc., according to various embodiments. In general, the historical weather data received in step 914 may include data from a time interval much greater than the time interval of the weather data received in step 906. For example, the weather data received in step 906 may include weather data collected over the course of the previous year, while the historical weather data received in step 914 may include weather data collected over the course of decades.

According to various embodiments, the historical weather data received in step 914 may be used to drive the building's energy consumption model, to determine energy use intensity values (EUIs) for the base case (step 918) and for the case in which FIMs have been implemented (step 916). If the same weather data (e.g. TMY3) is applied to both the base case and FIM inverse models, then the difference in predicted source EUI is attributed primarily to the FIMs. The inverse model predictions (Y) are normally distributed random variables and may be represented as follows:

$${Y}_{regression} = N ( X β , \frac{{ɛ}^{T} ɛ}{n - p - 1} ) the term \frac{{ɛ}^{T} ɛ}{n - p - 1} is equal to the square of the Standard Error , {SE}^{2}$$

where N signifies a normal random variable, Y is the vector of responses, X is the design or observation matrix, β is the coefficient vector, ε is the vector of model residuals or errors, n is the number of observations and p is the number of parameters in the model. The expected value of N is the first term and the variance σ² of N (second term) is approximated by SE².

If a normal random variable such as Y_{regression} is multiplied by a constant, the resulting random vector is as follows:

$$c \cdot {Y}_{regression} = N ( c \cdot X β , {c}^{2} \frac{{ɛ}^{T} ɛ}{n - p - 1} )$$

This operation would be used to convert from site to source energy consumption. Since the total energy consumption for a building may include both heating and cooling sources, the two random normal variables can be added together as shown below.

$${c}_{clg} {Y}_{clg} + {c}_{htg} {Y}_{htg} = {N ( {c}_{clg} \cdot {X}_{clg} {β}_{clg} + {c}_{htg} \cdot {X}_{htg} {β}_{htg} , {c}_{clg}^{2} \frac{{ɛ}_{clg}^{T} {ɛ}_{clg}}{n - p - 1} + {c}_{htg}^{2} \frac{{ɛ}_{htg}^{T} {ɛ}_{htg}}{n - p - 1} )}_{Base Model}$$

where the variables c_{clg} and c_{htg} represent site to source energy conversion factors. For example, if the cooling is done with electrical energy, the site to source conversion factor is 3.34. For natural gas it is 1.047.

The procedure described above can be repeated if a fixed deviation is applied to the model coefficients, as shown below:

$${Y}_{regression} = N ( X [ β + Δβ ] , \frac{{ɛ}^{T} ɛ}{n - p - 1} )$$

$$c \cdot {Y}_{regression =} N ( c \cdot X [ β + Δ β ] , {c}^{2} \frac{{ɛ}^{T} ɛ}{n - p - 1} )$$

$${c}_{clg} {Y}_{clg} + {c}_{htg} {Y}_{htg} = N ( {c}_{clg} \cdot {X}_{clg} [ {β}_{clg} + {Δβ}_{clg} ] + {c}_{htg} \cdot {X}_{htg} [ {β}_{htg} + {Δβ}_{htg} ] , {c}_{clg}^{2} \frac{{ɛ}_{clg}^{T} {ɛ}_{clg}}{n - p - 1} + {c}_{htg}^{2} \frac{{ɛ}_{htg}^{T} {ɛ}_{htg}}{n - p - 1} ) FIM Model$$

Since the variance term in the FIM model equations is identical to the variance term in the Base Model equations, shifting the model coefficients does not change the regression model variance. The source EUI equations for the base and FIMs cases can also be determined as follows:

$$E U {I}_{base} = {c}_{clg} \cdot {X}_{clg} {β}_{clg} + {c}_{htg} \cdot {X}_{htg} {β}_{htg} + {E}_{base}$$

$$E U {I}_{FIMs} = {c}_{clg} \cdot {X}_{clg} ⌊ {β}_{clg} + {Δβ}_{clg} ⌋ + {c}_{htg} \cdot {X}_{htg} ⌊ {β}_{htg} + Δ {β}_{htg} ⌋ + {E}_{FIMs}$$

$$where$$

$${E}_{base} = {E}_{FIMs} = N ( 0 , ( {c}_{clg}^{2} \frac{{ɛ}_{clg}^{T} {ɛ}_{clg}}{n - p - 1} + {c}_{htg}^{2} \frac{{ɛ}_{htg}^{T} {ɛ}_{htg}}{n - p - 1} ) I )$$

where the variable/is the identity matrix. Thus, the normalized random variables for the base case EUI (step 918) and FIM case EUI (step 916) as shown above.

Process 900 includes calculating an expected value of the ratio of EUIs (X_{EUI}) for the baseline and adjusted cases (step 920). In general, the implementation of FIMs typically does not impact the calculated source EUI since they do not change the building parameters (e.g., the building area, occupant working hours, worker density, weather, number of computers, etc.). Thus, the Energy Star model predictions would cancel out if the ER value for a building with a new FIM is divided by the ER for the building's base case (e.g., no FIMs):

$$\begin{matrix}{X}_{ER} ≡ \frac{E {R}_{FIMs}}{E {R}_{base}} \\ = \frac{Actual Source E U {I}_{FIMs}}{Predicted Source E U {I}_{FIMs}} / \frac{Actual Source E U {I}_{base}}{Predicted Source E U {I}_{base}} \\ = \frac{Actual Source E U {I}_{FIMs}}{Actual Source E U {I}_{base}}\end{matrix}$$

This is especially true for EUI_{base} and EUI_{FIMs} since they are highly correlated. For this particular problem however, it is possible to make a simplification which takes advantage of the predictable correlation [Δβ] between them, as shown below:

$$\begin{matrix}{X}_{EUI} = \frac{E U {I}_{FIMs}}{E U {I}_{base}} \\ = \frac{{c}_{clg} \cdot ( {X}_{clg} ⌊ {β}_{clg} + {Δβ}_{clg} ⌋ + {E}_{clg} ) + {c}_{htg} \cdot ( {X}_{htg} ⌊ {β}_{htg} + {Δβ}_{htg} ⌋ + {E}_{htg} )}{{c}_{clg} \cdot ( {X}_{clg} {β}_{clg} + {E}_{clg} ) + {c}_{htg} \cdot ( {X}_{htg} {β}_{htg} + {E}_{htg} )}\end{matrix}$$

which gives the following:

$${X}_{EUI} = \frac{E U {I}_{FIMs}}{E U {I}_{base}} = 1 + \frac{{c}_{clg} \cdot {X}_{clg} {Δβ}_{clg} + {c}_{htg} \cdot {X}_{htg} {Δβ}_{htg}}{E U {I}_{base} \sim N ( {μ}_{base} , {σ}_{base}^{2} )}$$

where:

μ_{base}=c_{clg}·└X_{clg}┘└β_{clg,base}┘+c_{htg}·└X_{htg}┘└β_{htg,base}┘

σ_{base}²=c_{clg}²·SE_{clg,base}²+c_{htg}²SE_{htg,base}².

X_{EUI} is the reciprocal of a normal random variable (EUI_{base}) multiplied by a scalar added to 1. The addition and multiplication will shift the expected value of the reciprocal of EUI_{base}. The probability density function (pdf) for the reciprocal of a normal random variable is derived below with the reciprocal being denoted as T:

$$E U {I}_{base} \sim N I D ( {μ}_{base} , {σ}_{base}^{2} )$$

$$T ≡ \frac{1}{E U {I}_{base} \sim N I D ( {μ}_{base} , {σ}_{base}^{2} )}$$

Calculation of the cumulative distribution function (cdf) for T requires the lower integration limit to be found as shown below. Next, the cdf of T must be differentiated to obtain the pdf of T. For notational convenience, EUI_{base} can be replaced with N for the remainder of the derivation:

cdf(T)=P(T<t)=P(1/N<t) therefore: 1/t<N.

As a result, the lower integration limit is 1/t for the transformed variable T. This limit is applied to the definite integral below to calculate the cdf for T:

$$c d f ( T ) = {\int }_{1 / t}^{∞} \frac{1}{σ \sqrt{2 π}} {e}^{- \frac{{( n - μ )}^{2}}{2 {σ}^{2}}} d n .$$

The integral and partial differential operators need to be interchanged and differentiation performed to obtain the pdf of T. This will also eliminate the random variable n as needed:

$$\frac{\partial c d f ( T )}{\partial T} = \frac{\partial }{\partial T} {\int }_{1 / t}^{∞} \frac{1}{σ \sqrt{2 π}} {e}^{\frac{- {( n - μ )}^{2}}{2 {σ}^{2}}} d n$$

Applying the Leibniz Integration Rule gives:

$$\frac{\partial c d f ( T )}{\partial T} = {\int }_{1 / t}^{∞} \frac{\partial }{\partial t} ( \frac{1}{σ \sqrt{2 π}} {e}^{\frac{- {( n - μ )}^{2}}{2 {σ}^{2}}} ) d n + \frac{1}{σ \sqrt{2 π}} {e}^{\frac{- {( ∞ - μ )}^{2}}{2 {σ}^{2}}} \cdot \frac{\partial ∞}{\partial t} - \frac{1}{σ \sqrt{2 π}} {e}^{\frac{- {( 1 / t - μ )}^{2}}{2 σ}} \cdot \frac{\partial ( \frac{1}{t} )}{\partial t}$$

where first two terms of the above equation are zero. Therefore, the pdf(T) is as follows:

$$p d f ( T ) = \frac{1}{{t}^{2} σ \sqrt{2 π}} {e}^{\frac{- {( \frac{1}{t} - μ )}^{2}}{2 {σ}^{2}}}$$

giving the expected value of T as:

$$E [ T ] = {\int }_{- ∞}^{∞} t \cdot p d f ( T ) d t = {\int }_{- ∞}^{∞} \frac{1}{t σ \sqrt{2 π}} {e}^{\frac{- {( \frac{1}{t} - μ )}^{2}}{2 {σ}^{2}}} d t .$$

An analytic solution to E[T] may not exist but it can be found using numerical integration techniques. Because E[T] is undefined at zero, care must be taken to avoid problems with the numerical integration. E[X_{EUI}] is obtained by shifting and scaling E[T] as described previously, leading to the following:

E[X_{EUI}]=1+(c_{clg}·└1η_{days/yr}CDD_{TMY/yr}┘└Δβ_{clg,}┘+c_{htg}·└1η_{days/yr}HDD_{TMY/yr}┘└Δβ_{htg}┘)·E[T]

An alternative approach to estimate the expected value of X_{EUI} is to assume that the elements of the ΔB vector are independent normally distributed random variables. The other variables are deterministic. The distribution of ΔB can be obtained by systematically introducing different FIMs into building energy simulations. Other analytical methods can also be used to determine ΔB. As shown below X_{EUI} is the sum of four normal random variables:

$$\begin{matrix}{X}_{EUI} = \frac{E U {I}_{base} + Δ E U {I}_{FIMs}}{E U {I}_{base}} \\ = 1 + \frac{{c}_{clg} {X}_{clg} {Δβ}_{FIM , clg} + {c}_{htg} {X}_{htg} {Δβ}_{FIM , htg}}{E U {I}_{base}}\end{matrix}$$

$${X}_{EUI} = 1 + \frac{{c}_{clg}}{E U {I}_{base}} ( {X}_{clg , 1} {Δβ}_{clg , 1} + {X}_{clg , 2} {Δβ}_{clg , 2} ) + \frac{{c}_{htg}}{E U {I}_{base}} ( {X}_{htg , 1} {Δβ}_{htg , 1} + {X}_{htg , 2} {Δβ}_{htg , 2} )$$

The expected value of X_{EUI} is then calculated as follows:

$$E [ {X}_{EUI} ] = 1 + \frac{{c}_{clg}}{E U {I}_{base}} ( {X}_{clg , 1} {μ}_{{Δβ}_{clg , 1}} + {X}_{clg , 2} {μ}_{{Δβ}_{clg , 2}} ) + \frac{{c}_{htg}}{E U {I}_{base}} ( {X}_{htg , 1} {μ}_{{Δβ}_{htg , 1}} + {X}_{htg , 2} {μ}_{{Δβ}_{htg , 2}} )$$

One advantage to this alternative approach is that it is simpler and computationally less intensive than the previously described approach based on the ratio of regression modeling results.

Process 900 includes receiving the Energy Star score for the baseline condition (step 922). As noted previously, the building's Energy Star score may be determined as follows:

Energy Star Score=Round(100*(1−gammaCDF(ER,α,β)))

where a cumulative gamma function is used on the building's ER value. The values of α, β are typically set as 5.6456 and 0.1741, respectively. However, different values of α, β may be used in other implementations.

Process 900 includes using the baseline Energy Star score to determine an ER value for the base case (step 924). In various embodiments, the ER value of the building in the base case (ER_{base}) is determined by applying an inverse gamma function to the building's Energy Star score. In other words, the ER value for the base case may be derived from the building's current Energy Star score.

Process 900 includes determining an adjusted ER value (step 926). According to various embodiments, the adjusted ER value that results from implementing FIMs, etc., may be determined using the ER value for the base case derived from the building's current Energy Star score (e.g., ER_{base}) in step 924 and the expected ratio of EUIs (e.g., E(X_{EUI}) determined in step 920. As noted previously, that the following holds true:

$${X}_{ER} ≡ \frac{{ER}_{FIMs}}{{ER}_{base}}$$

$$and$$

$${X}_{ER} \approx E [ {X}_{EUI} ] .$$

Thus, an adjusted ER value corresponding to the implementation of FIMs may be determined as follows:

ER_{FIMs}=ER_{base}·E[X_{EUI}].

Process 900 further includes calculating a new Energy Star score (step 928).

In one embodiment, the new Energy Star score may be calculated using the new ER value determined in step 926. For example, the predicted Energy Star score after implementing FIMs may be calculated as follows:

EnergyStar_{new}=Round(100*(1−gammaCDF(ER_{FIMs},alpha,beta)

where gammaCDF is the gamma function used to calculated the Energy Star score received in step 924 (e.g., the gamma function that corresponds to the inverse gamma function used in step 926), ER_{FIMs} is the new ER value calculated in step 926, alpha is a shape parameter for the gamma function, and beta is a scale parameter for the gamma function. In some embodiments, alpha may have a value of 5.6456 and beta may have a value of 0.1741. In other embodiments, alpha and beta may have values that correspond to those used in the inverse gamma function in step 924.

The resulting Energy Star score calculated in step 928 represents the predicted Energy Star score for the building that would result from the received action identifier. For example, assume that one action identifier corresponds to the building's chiller being upgraded to a more energy efficient model. Based on the building's current Energy Star score and the changes to the coefficients of the building's energy consumption model that result from the upgrade, a new Energy Star score for the building may be computed. In various embodiments, the updated Energy Star score may be reported to a user via an interface device (e.g., an electronic display, etc.), printer, or other device configured to convey information to a user. For example, the user may specify different action identifiers to review their predicted effects on the building's Energy Star score (e.g., by changing the action identifier received in step 910). In some embodiments, a received action identifier may be associated with multiple actions. For example, a particular action identifier may correspond to multiple equipment changes or the implementation of different ECMs. Thus, the user may also be able to pick and choose different combinations of actions to review their effects on the building's Energy Star score.

Configuration of Various Exemplary Embodiments

Embodiments of the subject matter and the operations described in this specification can be implemented in digital electronic circuitry, or in computer software embodied on a tangible medium, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on one or more computer storage medium for execution by, or to control the operation of, data processing apparatus. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate components or media (e.g., multiple CDs, disks, or other storage devices). Accordingly, the computer storage medium may be tangible and non-transitory.

The operations described in this specification can be implemented as operations performed by a data processing apparatus on data stored on one or more computer-readable storage devices or received from other sources.

The term “client or “server” include all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube), LCD (liquid crystal display), OLED (organic light emitting diode), TFT (thin-film transistor), plasma, other flexible configuration, or any other monitor for displaying information to the user and a keyboard, a pointing device, e.g., a mouse, trackball, etc., or a touch screen, touch pad, etc., by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an embodiment of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

While this specification contains many specific embodiment details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product embodied on a tangible medium or packaged into multiple such software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain embodiments, multitasking and parallel processing may be advantageous.

What is claimed is:

1. A method for evaluating a fault condition in a building comprising:
generating, by a processing circuit, an energy consumption model for the building;
using the energy consumption model and input data from different time windows to generate model data;
analyzing the model data to detect a non-routine change in the model data across the different time windows; and
providing an indication of a potential fault condition based on the non-routine change in the model data being detected.

2. The method of claim 1, wherein the input data comprises billing data from a utility that supplies energy to the building, and wherein the input data comprises weather data for the geographic area in which the building is located.

3. The method of claim 2, further comprising:
normalizing the model data by driving the energy consumption model using typical meteorological year (TMY) data to account for energy consumption changes attributable to routine weather changes.

4. The method of claim 1, further comprising:
using the generated model data to train a control chart having control limits based on the model data, wherein the non-routine change in the model data is detected by comparing model data associated with a new time window to the control limits of the control chart.

5. The method of claim 4, wherein the control chart is an exponentially weighted moving average (EWMA) control chart.

6. The method of claim 4, wherein the control chart comprises at least one of: a moving average control chart, an Xbar control chart, a Shewhart control chart, or a cumulative sum control chart.

7. The method of claim 1, further comprising:
receiving a test observation corresponding to model data from a new time window; and
generating a confidence interval for a point estimate based on the model data, wherein the non-routine change in the model data is detected by comparing model data associated with a new time window to the control limits of the control chart.

8. The method of claim 1, further comprising:
using a null-hypothesis test to detect the non-routine change in the model data.

9. The method of claim 1, further comprising:
calculating one or more recursive residual values using the model data; and
analyzing the one or more recursive residual values to detect the non-routine change in the model data.

10. The method of claim 9, wherein the one or more recursive residual values are analyzed using a statistical process control chart.

11. The method of claim 10, wherein the control chart is an exponentially weighted moving average (EWMA) control chart.

12. The method of claim 9, wherein the one or more recursive residual values are analyzed using a cumulative sum test or a cumulative sum of squares test.

13. A system for evaluating a fault condition in a building comprising a processing circuit configured to generate an energy consumption model for the building, wherein the processing circuit is configured to use the energy consumption model and input data from different time windows to generate model data, wherein the processing circuit is configured to analyze the model data to detect a non-routine change in the model data across the different time windows, and wherein the processing circuit is configured to provide an indication of a potential fault condition based on the non-routine change in the model data being detected.

14. The system of claim 13, wherein the input data comprises billing data from a utility that supplies energy to the building, and wherein the input data comprises weather data for the geographic area in which the building is located.

15. The system of claim 14, wherein the processing circuit is configured to normalize the model data by driving the energy consumption model using typical meteorological year (TMY) data to account for energy consumption changes attributable to routine weather changes.

16. The system of claim 13, wherein the processing circuit is configured to use the generated model data to train a control chart having control limits based on the model data, wherein the non-routine change in the model data is detected by comparing model data associated with a new time window to the control limits of the control chart.

17. The system of claim 16, wherein the control chart is an exponentially weighted moving average (EWMA) control chart.

18. The system of claim 16, wherein the control chart comprises at least one of: a moving average control chart, an Xbar control chart, a Shewhart control chart, or a cumulative sum control chart.

19. The system of claim 13, wherein the processing circuit is configured to generate a confidence interval for a point estimate based on the model data, wherein the non-routine change in the model data is detected by comparing model data associated with a new time window to the control limits of the control chart.

20. The system of claim 13, wherein the processing circuit is configured to use a null-hypothesis test to detect the non-routine change in the model data.

21. The system of claim 13, wherein the processing circuit is configured to calculate one or more recursive residual values using the model data, wherein the processing circuit is configured to analyze the one or more recursive residual values to detect the non-routine change in the model data.

22. The system of claim 21, wherein the one or more recursive residual values are analyzed using a statistical process control chart.

23. The system of claim 22, wherein the control chart is an exponentially weighted moving average (EWMA) control chart.

24. The system of claim 21, wherein the one or more recursive residual values are analyzed using a cumulative sum test or a cumulative sum of squares test.

25. A method for determining a change to an energy score of a building comprising:
generating, by a processing circuit, an energy consumption model for the building;
using the energy consumption model and input data regarding the building to calculate baseline model data, the baseline model data being associated with a baseline energy score;
receiving an identifier representing a proposed change to the operation of the building, the received identifier being associated with a change to the model data; and
calculating an energy score associated with the proposed change using the baseline model data, the change to the model data associated with the proposed change, and the baseline energy score.

26. The method of claim 25, wherein the energy score comprises an Energy Star score associated with the proposed change.

27. The method of claim 26, further comprising:
normalizing the baseline model data using typical meteorological year (TMY) data to determine a baseline normalized annual consumption intensity value;
using the change to the model data associated with the received identifier and the TMY data to determine a normalized annual consumption intensity value associated with the proposed change;
calculating an energy use intensity ratio relating the baseline normalized annual consumption energy intensity value to the normalized annual consumption intensity value associated with the proposed change; and
using the energy use intensity ratio to calculate the Energy Star score associated with the proposed change.

28. The method of claim 27, further comprising:
calculating a baseline energy efficiency ratio for the building;
calculating an energy efficiency ratio associated with the proposed change using the baseline energy efficiency ratio and the energy use intensity ratio; and
using the energy efficiency ratio associated with the proposed change to calculate the Energy Star score associated with the proposed change.

29. The method of claim 28, further comprising:
using an inverse gamma function to calculate the baseline energy efficiency ratio.

30. The method of claim 29, further comprising:
using the energy efficiency ratio associated with the proposed change with a gamma function to calculate the Energy Star score associated with the proposed change.

31. The method of claim 25, wherein the proposed change to the operation of the building comprises at least one of: implementing an energy conservation measure or altering equipment in the building.
